(19) United States
(12) Patent Application Publication
(10) Pub. No.: US 2026/0085348 A1
(43) Pub. Date: Mar. 26, 2026

(54) UNIQUE MOLECULAR IDENTIFIER ENHANCED HLA GENOTYPING AND TRANSCRIPT QUANTITATION USING NANOPORE TECHNOLOGY

(71) Applicant: The University of North Carolina at Chapel Hill, Chapel Hill, NC (US)

(72) Inventors: Eric Thomas Weimer, Durham, NC (US); Maureen C. Montgomery, Pittsboro, NC (US)

(73) Assignee: The University of North Carolina at Chapel Hill, Chapel Hill, NC (US)

(21) Appl. No.: 18/866,905

(22) PCT Filed: May 18, 2023
(86) PCT No.: PCT/US23/67187

§ 371 Date: Nov. 18, 2024

Related U.S. Application Data
(60) Provisional application No. 63/343,473, filed on May 18, 2022.

(51) Int. Cl. C12Q 1/6869; C12N 15/10; C12Q 1/6806; C12Q 1/6881

(52) U.S. Cl. CPC C12Q 1/6869; C12N 15/1096; C12Q 1/6806; C12Q 1/6881; C12Q 2600/158

(57) ABSTRACT

Provided are methods for rapid multiplex HLA genotyping and transcript quantitation. In some embodiments, the presently disclosed methods include providing a. sample from the cell, tissue, or organ that has mRNA derived from an HLA gene product; reverse transcribing the mRNA to produce a. pool of cDNAs; amplifying the cDNAs that result from reverse transcription of HLA-specific mRNAs to produce a pool of HLA-specific cDNAs; and determining the sequence of each HLA-specific cDNAs, whereby the HLA status of the cell, tissue, organ, or subject is determined. Also provided are methods for genotyping donor cells, tissues, and/or organs meant for transplantation into recipients. In some embodiments, the methods can be used to identify the presence of absence of each of HLA-A, -B, -C, -DRB 1/3/4/5, -DQA1, and -DQB1 in a biological sample.

### REFERENCE TO SEQUENCE LISTING XML SUBMITTED ELECTRONICALLY

The Sequence Listing XML associated with the instant disclosure has been electronically submitted to the United States Patent and Trademark Office via the Patent Center as a 42,999 byte UTF-8-encoded XML file created on May 18, 2023 and entitled “421_514_PCT.xml”. The Sequence Listing submitted via Patent Center is hereby incorporated by reference in its entirety.

### TECHNICAL FIELD

The presently disclosed subject matter relates generally to methods for multiplex HLA genotyping and transcript quantitation, particularly with respect to rapid, multiplex determination of HLA status of cells, tissues, organs, and/or subjects. The presently disclosed subject matter also relates to methods for genotyping donor cells, tissues, and/or organs meant for transplantation into a recipient.

### BACKGROUND

The application of next-generation sequencing (NGS) to human leukocyte antigen (HLA) typing has greatly reduced the costs and time required to achieve high-resolution HLA typing (Lind et al., 2010; Weimer et al., 2016; Cornaby et al., 2021). Currently, there are two major NGS platforms: short and long-read sequencing. Short read platforms are characterized by sequencing reads between 50 and 300 bp, while long-read platforms can sequence reads from 150 to >2 Mb (Jain et al., 2018; Metzker, 2010). The application of long-read technology to HLA typing has been successfully demonstrated by several laboratories using DNA or RNA (De Santis et al., 2020; Lang et al., 2018; Liu et al., 2018a; Liu et al., 2021; Montgomery et al., 2019; Mosbruger et al., 2020). Long read technology is beneficial and applicable for HLA typing due to various advantages of the methodology, including shorter library preparation, real-time base calling, shorter sequencing times, and potential cost savings. The main focus for rapid HLA typing has been the development of an assay that yields high-resolution HLA typing in the time necessary for deceased donor allocation. Such an assay would enable improved virtual crossmatching and epitope analysis (Garcia-Sanchez et al., 2020).

A challenge with Oxford Nanopore Technologies (ONT) sequencers is the relatively higher error rate compared to traditional short-read sequencers. Short read platforms obtain reads with an accuracy approaching 99.99%; however, ONT sequencers achieve between 88-94% accuracy, depending on the chemistry and flow cell used (Liu et al., 2021; Metzker, 2010). One approach to reducing raw sequencing errors with ONT sequencing is the attachment of random oligomers of DNA to DNA or RNA before PCR amplification. The incorporation of unique molecular identifiers (UMIs) in the initial library preparation has become standard for single-cell analysis (Chen et al., 2018; Islam et al., 2014). Application of UMI to a library enables several benefits including removal of PCR bias (Islam et al., 2014), quantitation of input molecules (Islam et al., 2014; Kivioja et al., 2011), and increased consensus read accuracy (Kou et al., 2016).

There is growing evidence for the importance of HLA expression in various diseases (Dendrou et al., 2018; Cornaby & Weimer, 2020; Cornaby et al., 2021) and allele-specific expression that influences T cell activation (Gutierrez-Arcelus et al., 2020). Allele-specific HLA expression has been studied from single-cell sequencing data (Darby et al., 2020), and Johansson et al., 2021 using short-read sequencing found variable allele-specific HLA mRNA expression. HLA-DPB1 expression has been shown to influence graft versus host disease (Apps et al., 2013; Petersdorf et al., 2015). Recently. HLA expression has been shown to impact solid organ recipient and deceased donor immunologic compatibility (Badders et al., 2015; Montgomery et al., 2019). However, there are no current approaches to assess allele-specific HLA expression, protein, or transcript on deceased donors before transplantation. With allele-specific expression of donor antigens, virtual crossmatch predictions could be more accurate, and patient risk stratification could be assessed with more granularity.

Described herein is an assay that provides high-resolution HLA typing and allele-specific HLA expression within the time constraints for deceased organ donor allocation. Using RNA and the application of UMI to quantify the mRNA HLA expression, the data demonstrate the variability of individual HLA allele groups.

### SUMMARY

This Summary lists several embodiments of the presently disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without one or more of the features mentioned; likewise, those features can be applied to other embodiments of the presently disclosed subject matter, whether listed in this Summary or not. To avoid excessive repetition, this Summary does not list or suggest all possible combinations of such features.

The presently disclosed subject matter provides in some embodiments methods for rapid, multiplex determination of HLA status of a cell, tissue, organ, or subject. In some embodiments, the method comprises, consists essentially of, or consists of (a) providing a sample from the cell, tissue, or organ that comprises mRNA derived from an HLA gene product; (b) reverse transcribing the mRNA to produce a pool of cDNAs, wherein the pool of cDNAs comprises a plurality of HLA-specific cDNAs; (c) amplifying the cDNAs that result from reverse transcription of HLA-specific mRNAs to produce a pool of HLA-specific cDNAs; and (d) determining the sequence of each HLA-specific cDNAs, whereby the HLA status of the cell, tissue, organ, or subject is determined. In some embodiments, the HLA-specific mRNAs are transcription products of one or more of HLA-A, -B, -C, -E, -F, G, -H, -DRB1, -DRB3, -DRB4, -DRB5, -DQA1, -DQB1, -DPA1, -DPB1, MICA, and MICB loci, optionally for each of at least HLA-A, -B, -C, -DRB1/3/4/5, -DQA1, and -DQB1. In some embodiments, the amplifying comprises an enrichment step and an amplification step, wherein the amplification step is performed subsequent to the enrichment step. In some embodiments, the amplification employs HLA-specific primers that comprise unique molecular identifiers (UMIs) such that the HLA-specific cDNAs are each uniquely coded. In some embodiments, both members of a particular PCR primer pair comprises a UMI, and in some embodiments both members of a particular PCR primer pair comprises a UMI. In some embodiments, the sample is a blood sample. In some embodiments, the sample is isolated from a deceased donor. In some embodiments, the amplifying identifies each of HLA-A, -B, -C, -DRB1/3/4/5, -DQA1, and -DQB1 if present in the sample. In some embodiments, the HLA-specific primers comprise HLA-specific sequences, and further wherein the HLA-specific sequences are selected from the group consisting of SEQ ID NO: 1 and SEQ ID NO: 2 for HLA-A, SEQ ID NO: 3 and SEQ ID NO: 4 for HLA-B, SEQ ID NO: 5 and SEQ ID NO: 6 for HLA-C, SEQ ID NO: 7 and SEQ ID NO: 8 for HLA-DRB1/3/4/5, SEQ ID NO: 9 SEQ ID NO: 10 for HLA-DPA1, SEQ ID NO: 11 SEQ ID NO: 12 for HLA-DPB1, SEQ ID NO: 13 and SEQ ID NO: 14 for HLA-DQA1, and SEQ ID NO: 15 and SEQ ID NO: 16 for HLA-DQB1.

In some embodiments of the presently disclosed methods, the determining the sequence of each HLA-specific cDNAs step comprises use of an Oxford Nanopore Technologies flow cell, optionally selected from the group consisting of an R10.3 flow cell, an R10.4 flow cell, an R9.4 flow cell, and a Flongle flow cell, and an Oxford Nanopore Technologies MinION R10.3 Flow Cell sequencer, optionally selected from the group consisting of a MinION sequencer, a MinION Mk1C sequencer, a Promethion sequencer, a Gridion sequencer, a MinION Mk1D sequencer, a SmidgION sequencer, and a Plongle sequencer.

In some embodiments of the presently disclosed methods, the cell, tissue, or organ is a donor cell, tissue, or organ meant for transfer to a recipient or is derived from a donor cell, tissue, or organ meant for transfer to a recipient.

In some embodiments, the determination of HLA status of the cell, tissue, organ, or subject is intended for use in target point of care viral load testing to provide absolute viral counts, optionally wherein the virus is an HIV or a CMV, or is intended for use in diagnosis of an immune-associated disease, disorder, or condition, optionally wherein the immune-associated disease, disorder, or condition is Bechet Disease, Celiac Disease, or ankylosing spondylitis.

In some embodiments, the presently disclosed subject matter also related to methods for genotyping donor cells, tissues, and/or organs meant for transplantation into recipients. In some embodiments, the methods comprise, consist essentially of, or consist of providing a donor cell, tissue, and/or organ meant for transplantation into a recipient that comprises mRNA derived from an HLA gene product; reverse transcribing the mRNA to produce a pool of cDNAs, wherein the pool of cDNAs comprises a plurality of HLA-specific cDNAs; amplifying the cDNAs that result from reverse transcription of HLA-specific mRNAs to produce a pool of HLA-specific cDNAs, wherein (i) the HLA-specific mRNAs are transcription products of one or more of HLA-A, HLA-B, HLA-C, HLA-E, HLA-F, HLA-G, HLA-H, HLA-DRB1, HLA-DRB3, HLA-DRB4, HLA-DRB5, HLA-DQA1, HLA-DQB1, HLA-DPA1, HLA-DPB1, MICA, and MICB loci, optionally for each of at least HLA-A, HLA-B, HLA-C, HLA-DRB1/3/4/5, HLA-DQA1, and HLA-DQB1; (ii) the amplifying comprises an enrichment step and an amplification step, wherein the amplification step is performed subsequent to the enrichment step; and (iii) the amplification employs HLA-specific primers that comprise unique molecular identifiers (UMIs) such that the HLA-specific cDNAs are each uniquely coded; and determining the sequence of each HLA-specific cDNAs, whereby the donor cell, tissue, and/or organ meant for transplantation into a recipient is genotyped. In some embodiments, the donor cell, tissue, and/or organ is a cadaveric cell, tissue, and/or organ. In some embodiments, the amplifying identifies each of HLA-A, -B, -C, -DRB1/3/4/5, -DQA1, and -DQB1 if present in the sample. In some embodiments, the HLA-specific primers comprise HLA-specific sequences, and further wherein the HLA-specific sequences comprise sequences selected from the group consisting of SEQ ID NO: 1 and SEQ ID NO: 2 for HLA-A, SEQ ID NO: 3 and SEQ ID NO: 4 for HLA-B, SEQ ID NO: 5 and SEQ ID NO: 6 for HLA-C, SEQ ID NO: 7 and SEQ ID NO: 8 for HLA-DRB1/3/4/5, SEQ ID NO: 9 SEQ ID NO: 10 for HLA-DPA1, SEQ ID NO: 11 SEQ ID NO: 12 for HLA-DPB1, SEQ ID NO: 13 and SEQ ID NO: 14 for HLA-DQA1, and SEQ ID NO: 15 and SEQ ID NO: 16 for HLA-DQB1, the determining the sequence of each HLA-specific cDNAs step comprises use of an Oxford Nanopore Technologies flow cell, optionally selected from the group consisting of an R10.3 flow cell, an R10.4 flow cell, an R9.4 flow cell, and a Flongle flow cell, and an Oxford Nanopore Technologies MinION R10.3 Flow Cell sequencer, optionally selected from the group consisting of a MinION sequencer, a MinION Mk1C sequencer, a Promethion sequencer, a Gridion sequencer, a MinION Mk1D sequencer, a SmidgION sequencer, and a Plongle sequencer.

Thus, it is an object of the presently disclosed subject matter to provide methods for rapid multiplex HLA genotyping and transcript quantitation. This and other objects are achieved in whole or in part by the presently disclosed subject matter.

Further, an object of the presently disclosed subject matter having been stated above, other objects and advantages of the presently disclosed subject matter will become apparent to those skilled in the art after a study of the following description, Figures, and Examples.

### BRIEF DESCRIPTION OF THE FIGURES

FIG. 1. Patient sample workflow for RNA isolation, library preparation, and resultant HLA typing as described based on consensus sequence generation. In short, total RNA was isolated from peripheral blood and a portion of the total RNA was purified. RNA fragmentation and quantitation were determined and an RNA integrity number (RIN) was calculated for each sample. cDNA synthesis was then performed after which PCR amplification and purification were utilized to achieve the final enriched HLA-locus specific UMI tagged amplicon. The HLA gene-specific amplicons for each patient were then used for library preparation and bar coding with the Oxford Nanopore Technologies (ONT) platform. Sequencing was performed using the MinION R10.3 Flow Cell and loaded on the MinION Mk1C using the high-accuracy base calling module. The NGSEngine bioinformatics pipeline was then utilized to provide HLA typing and Athlon2 was used to generate HLA allele-specific UMI counts.

FIGS. 2A-2H. Metrics for HLA typing using unique molecular identifier-tagged RNA demonstrated that as fragmentation decreases, more reads are obtained for sequencing, and a greater proportion of reads can be mapped to HLA loci of interest. (FIG. 2A) Calculated RNA integrity numbers for each patient sample with their measured band density post isolation and clean-up. (FIG. 2B) Sample sequencing reads acquired and the proportion of the reads that contain recognized UMI barcodes. (FIG. 2C) The proportion of reads per each classical HLA locus per patient sample as counted using UMI reads. (FIG. 2D) The proportion of reads for each HLA locus compared to the calculated RNA integrity number for each patient sample. (FIG. 2E) The percentage of cDNA reads obtained that could be mapped to the HLA loci obtained during sequencing and their respective calculated sample RNA integrity number. (FIG. 2F) Read accuracy calculated for the minimum number of UMIs required for each HLA locus and sample after concatenation of cDNA reads to consensus sequences. The percentage of class I (FIG. 2G) and class II (FIG. 2H) HLA specific reads mapped compared to input isolated RNA measured band density (****P value<0.001).

FIGS. 3A-3D. RNA expression of the patient cohort as counted by unique molecular identifier. (FIG. 3A) The normalized UMI count distribution for each patient sample corresponding to the calculated RNA integrity number. (FIG. 3B) The distribution of the normalized UMI counts across several representative patient HLA alleles and their corresponding calculated RNA integrity number. All 9 of these patient samples were used to evaluate interassay precision, and these data are reflected on the respective patient plots. (FIG. 3C) Normalized UMI counts at each of the classical HLA loci (*P value<0.05, **P value<0.01). (FIG. 3D) The distribution of the UMI counts obtained for each HLA allele typed for the classical HLA loci for the study patient cohort.

FIGS. 4A-4C. Sample and HLA loci mRNA transcript counts ae extremely similar even after doubling the concentration of input cDNA. Half of the cohort samples had the cDNA input doubled and the assay repeated. The graphs illustrate the (FIG. 4A) normalized transcript levels observed for each patient sample, (FIG. 4B) each HLA allele, and (FIG. 4C) each of the classical HLA loci investigated (*p value<0.05).

FIG. 5. Impact of varying unique molecular identifier primers on transcript expression. Two of the samples were tested using the RNA-Seq HLA typing assay described utilizing varying amounts of assay UMI primers for each HLA locus (x-axis). These results compare the expression (y-axis) calculated for these samples for each HLA allele. Paired analysis was performed using the Wilcoxon paired test (ns: not statistically significant).

### BRIEF DESCRIPTION OF THE SEQUENCE LISTING

SEQ ID NOs: 1 and 2 are exemplary nucleotide sequences that can be employed together for amplifying HLA-A-specific genes and gene products.

SEQ ID NOs: 3 and 4 are exemplary nucleotide sequences that can be employed together for amplifying HLA-B-specific genes and gene products.

SEQ ID NOs: 5 and 6 are exemplary nucleotide sequences that can be employed together for amplifying HLA-C-specific genes and gene products.

SEQ ID NOs: 7 and 8 are exemplary nucleotide sequences that can be employed together for amplifying HLA-DRB1/3/4/5-specific genes and gene products.

SEQ ID NOs: 9 and 10 are exemplary nucleotide sequences that can be employed together for amplifying HLA-DPA1-specific genes and gene products.

SEQ ID NOs: 11 and 12 are exemplary nucleotide sequences that can be employed together for amplifying HLA-DPB1-specific genes and gene products.

SEQ ID NOs: 13 and 14 are exemplary nucleotide sequences that can be employed together for amplifying HLA-DQA1-specific genes and gene products.

SEQ ID NOs: 15 and 16 are exemplary nucleotide sequences that can be employed together for amplifying HLA-DQB1-specific genes and gene products.

SEQ ID NO: 17 is the nucleotide sequence of a consensus GSP UMI Forward Primer shown in Table 2. In SEQ ID NO: 17, “V” refers to a nucleotide other than T (i.e., A, C, or G) and the “nnnnnnnnnnnnnnnnnnn” nucleotides correspond to one of the Forward Primers listed in Table 3 (i.e., one of SEQ ID NOs: 1, 3, 5, 7, 9, 11, 13, and 15).

SEQ ID NOs: 18-25 are the nucleotide sequences of SEQ ID NO: 17 complexed to SEQ ID NO: 1, 3, 5, 7, 9, 11, 13, and 15, respectively.

SEQ ID NO: 26 is the nucleotide sequence of a consensus GSP UMI Reverse Primer shown in Table 2. In SEQ ID NO: 26, “V” refers to a nucleotide other than T (i.e., A, C, or G) and the “nnnnnnnnnnnnnnnnnnn” nucleotides correspond to one of the Reverse Primers listed in Table 3 (i.e., one of SEQ ID NOs: 2, 4, 6, 8, 10, 12, 14, and 16).

SEQ ID NOs: 27-34 are the nucleotide sequences of SEQ ID NO: 26 complexed to SEQ ID NO: 2, 4, 6, 8, 10, 12, 14, and 16, respectively.

SEQ ID NOs: 35 and 36 are the nucleotide sequences of the UVP Forward and Reverse primers shown in Table 2.

### DETAILED DESCRIPTION

### I. General Considerations

HLA typing provides essential results for stem cell and solid organ transplants, as well as providing diagnostic benefits for various rheumatology, gastroenterology, neurology, and infectious diseases. It is becoming increasingly clear that understanding the expression of patient HLA transcripts can provide additional benefits for many of these same patient groups. The presently disclosed study cohort was evaluated using a long-read RNA sequencing methodology to provide rapid HLA genotyping results and normalized HLA transcript expression. The presently disclosed assay used NGSEngine to determine the HLA genotyping result and normalized mRNA transcript expression using Athlon2. The assay demonstrated an excellent concordance rate of 99.7%. Similar to previous studies, for the class I loci, patients demonstrated significantly lower expression of HLA-C than HLA-A and -B (Mann-Whitney U, p value=0.0065 and p value=0.0154, respectively). In general, the expression of class II transcripts was lower than that of class I transcripts. The presently disclosed subject matter thus demonstrated a rapid high-resolution HLA typing assay using RNA-Seq that can provide accurate HLA genotyping and HLA allele-specific transcript expression in 7 to 8 hours, a timeline short enough to perform the assay for deceased donors.

### II. Definitions

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent techniques that would be apparent to one of skill in the art. While the following terms are believed to be well understood by one of ordinary skill in the art, the following definitions are set forth to facilitate explanation of the presently disclosed subject matter.

Following long-standing patent law convention, the terms “a”, “an”, and “the” mean “one or more” when used in this application, including the claims. Thus, the phrase “a cell” refers to one or more cells, unless the context clearly indicates otherwise.

As used herein, the term “and/or” when used in the context of a list of entities, refers to the entities being present singly or in combination. Thus, for example, the phrase “A, B, C, and/or D” includes A, B, C, and D individually, but also includes any and all combinations and subcombinations of A, B, C, and D.

The term “comprising”, which is synonymous with “including”, “containing”, and “characterized by”, is inclusive or open-ended and does not exclude additional, unrecited elements and/or method steps. “Comprising” is a term of art that means that the named elements and/or steps are present, but that other elements and/or steps can be added and still fall within the scope of the relevant subject matter.

As used herein, the phrase “consisting of” excludes any element, step, and/or ingredient not specifically recited. For example, when the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of the related disclosure or claim to the specified materials and/or steps, plus those that do not materially affect the basic and novel characteristic(s) of the disclosed and/or claimed subject matter.

With respect to the terms “comprising”, “consisting essentially of”, and “consisting of”, where one of these three terms is used herein, the presently disclosed and claimed subject matter can include the use of either of the other two terms. For example, it is understood that the methods of the presently disclosed subject matter in some embodiments comprise the steps that are disclosed herein and/or that are recited in the claims, in some embodiments consist essentially of the steps that are disclosed herein and/or that are recited in the claims, and in some embodiments consist of the steps that are disclosed herein and/or that are recited in the claim.

The term “subject” as used herein refers to a member of any invertebrate or vertebrate species. Accordingly, the term “subject” is intended to encompass any member of the Kingdom Animalia including, but not limited to the phylum Chordata (i.e., members of Classes Osteichythyes (bony fish), Amphibia (amphibians), Reptilia (reptiles), Aves (birds), and Mammalia (mammals)), and all Orders and Families encompassed therein. In some embodiments, the presently disclosed subject matter relates to human subjects.

Similarly, all genes, gene names, and gene products disclosed herein are intended to correspond to orthologs from any species for which the compositions and methods disclosed herein are applicable. Thus, the terms include, but are not limited to genes and gene products from humans. It is understood that when a gene or gene product from a particular species is disclosed, this disclosure is intended to be exemplary only, and is not to be interpreted as a limitation unless the context in which it appears clearly indicates. Thus, for example, the genes and/or gene products disclosed herein are also intended to encompass homologous genes and gene products from other animals including, but not limited to other mammals, fish, amphibians, reptiles, and birds.

The methods and compositions of the presently disclosed subject matter are particularly useful for warm-blooded vertebrates. Thus, the presently disclosed subject matter concerns mammals and birds. More particularly provided is the use of the methods and compositions of the presently disclosed subject matter on mammals such as humans and other primates, as well as those mammals of importance due to being endangered (such as Siberian tigers), of economic importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), rodents (such as mice, rats, and rabbits), marsupials, and horses. Also provided is the use of the disclosed methods and compositions on birds, including those kinds of birds that are endangered, kept in zoos, as well as fowl, and more particularly domesticated fowl, e.g., poultry, such as turkeys, chickens, ducks, geese, guinea fowl, and the like, as they are also of economic importance to humans. Thus, also provided is the application of the methods and compositions of the presently disclosed subject matter to livestock, including but not limited to domesticated swine (pigs and hogs), ruminants, horses, poultry, and the like.

The term “about”, as used herein when referring to a measurable value such as an amount of weight, time, dose, etc., is meant to encompass variations of in some embodiments ±20%, in some embodiments ±10%, in some embodiments ±5%, in some embodiments ±1%, and in some embodiments ±0.1% from the specified amount, as such variations are appropriate to perform the disclosed methods and/or to employ the presently disclosed arrays.

As used herein the term “gene” refers to a hereditary unit including a sequence of DNA that occupies a specific location on a chromosome and that contains the genetic instruction for a particular characteristic or trait in an organism. Similarly, the phrase “gene product” refers to biological molecules that are the transcription and/or translation products of genes. Exemplary gene products include, but are not limited to mRNAs and polypeptides that result from translation of mRNAs. Any of these naturally occurring gene products can also be manipulated in vivo or in vitro using well known techniques, and the manipulated derivatives can also be gene products. For example, a cDNA is an enzymatically produced derivative of an RNA molecule (e.g., an mRNA), and a cDNA is considered a gene product. Additionally, polypeptide translation products of mRNAs can be enzymatically fragmented using techniques well known to those of skill in the art, and these peptide fragments are also considered gene products.

The term “isolated”, as used in the context of a nucleic acid or polypeptide (including, for example, a nucleotide sequence, a polypeptide, and/or a peptide), indicates that the nucleic acid or polypeptide exists apart from its native environment. An isolated nucleic acid or polypeptide can exist in a purified form or can exist in a non-native environment.

Further, as used for example in the context of a cell, nucleic acid, polypeptide, or peptide, the term “isolated” indicates that the cell, nucleic acid, polypeptide, or peptide exists apart from its native environment. In some embodiments, “isolated” refers to a physical isolation, meaning that the cell, nucleic acid, polypeptide, or peptide has been removed from its native environment (e.g., from a subject).

The terms “nucleic acid molecule” and “nucleic acid” refer to deoxyribonucleotides, ribonucleotides, and polymers thereof, in single-stranded or double-stranded form. Unless specifically limited, the term encompasses nucleic acids containing known analogues of natural nucleotides that have similar properties as the reference natural nucleic acid. The terms “nucleic acid molecule” and “nucleic acid” can also be used in place of“gene”. “cDNA”, and “mRNA”, Nucleic acids can be synthesized, or can be derived from any biological source, including any organism.

As used herein, the terms “peptide” and “polypeptide” refer to polymers of at least two amino acids linked by peptide bonds. Typically, “peptides” are shorter than “polypeptides”, but unless the context specifically requires, these terms are used interchangeably herein.

As used herein, a cell, nucleic acid, or peptide exists in a “purified form” when it has been isolated away from some, most, or all components that are present in its native environment, but also when the proportion of that cell, nucleic acid, or peptide in a preparation is greater than would be found in its native environment. As such, “purified” can refer to cells, nucleic acids, and peptides that are free of all components with which they are naturally found in a subject, or are free from just a proportion thereof.

### II. Exemplary Embodiments

As is known, there are many HLA genes/alleles that play a role in acceptance of donor cells, tissues, and organs to recipients. As such, it can be important to identify the HLA status of donor cells, tissues, and organs to ensure that the cells, tissues, and organs that might be transferred to a recipient will not induce anti-donor immune responses such as but not limited to host vs. graft disease. In the case of donor cells, tissues, and organs that include immune cells, this can also be important in order to avoid graft vs. host disease.

Thus, in some embodiments the presently disclosed subject matter relates to methods for rapid, multiplex determination of HLA status of cells, tissues, organs, and/or subjects. In some embodiments, the methods comprise, consist essentially of, or consist of providing a sample from the cell, tissue, or organ that comprises mRNA derived from an HLA gene product; reverse transcribing the mRNA to produce a pool of cDNAs, wherein the pool of cDNAs comprises a plurality of HLA-specific cDNAs; amplifying the cDNAs that result from reverse transcription of HLA-specific mRNAs to produce a pool of HLA-specific cDNAs; and determining the sequence of each HLA-specific cDNAs, whereby the HLA status of the cell, tissue, organ, and/or subject is determined.

Exemplary HLA-specific mRNAs are transcription products include those that encode one or more of the HLA-A, HLA-B, HLA-C, HLA-E HLA-F, HLA-G, HLA-H, HLA-DRB, HLA-DRB3, HLA-DRB4, HLA-DRB5, HLA-DQA1, HLA-DQB, HLA-DPA1, HLA-DPB1, MICA, and MICB gene products. Nucleotide and amino acid sequences of various genes and gene products are set forth in the following Accession Numbers of the GENBANK® biosequence database as follows:

| | | Transcript | Protein |
|---|---|---|---|
| Locus | Genomic* | Accession Number(s) | Accession Number(s) |
| HLA-A | 29,942,532-29,945,870 | NM_001242758.1 | NP_001229687.1 |
| | | NM_002116.8 | NP_002107.3 |
| HLA-B | 31,353,875-31,357,179** | NM_005514.8 | NP_005505.2 |
| HLA-C | 31,268,749-31,272,092** | NM_001243042.1 | NP_001229971.1 |
| | | NM_002117.6 | NP_002108.4 |
| HLA-E | 30,489,509-30,494,194 | NM_005516.6 | NP_005507.3 |
| HLA-F | 29,723,434-29,738,532 | NM_001098479.2 | NP_001091949.1 |
| | | NM_018950.3 | NP_061823.2 |
| | | NM_001098478.2 | NP_001091948.1 |
| HLA-G | 29,826,474-29,831,021 | NM_001363567.2 | NP_001350496.1 |
| | | NM_001384280.1 | NP_001371209.1 |
| | | NM_001384290.1 | NP_001371219.1 |
| | | NM_002127.6 | NP_002118.1 |
| HLA-H | 29,887,573-29,891,079 | | |
| HLA-DRB1 | 32,578,775-32,589,848** | NM_001243965.1 | NP_001230894.1 |
| | | NM_001359193.1 | NP_001346122.1 |
| | | NM_001359194.1 | NP_001346123.1 |
| | | NM_002124.4 | NP_002115.2 |
| HLA-DRB3 | 32,578,772-32,589,782 | NM_022555.3 | NP_072049.2 |
| HLA-DRB4 | 32,530,295-32,552,722** | NM_021983.4 | NP_068818.4 |
| HLA-DRB5 | 32,517,353-32,530,287** | NM_002125.4 | NP_002116.2 |
| HLA-DQA1 | 32,637,406-32,655,272 | NM_002122.5 | NP_002113.2 |
| HLA-DQB1 | 32,659,880-32,660,729 | | |
| HLA-DPA1 | 33,064,569-33,080,748** | NM_001242524.2 | NP_001229453.1 |
| | | NM_001242525.2 | NP_001229454.1 |
| | | NM_033554.3 | NP_291032.2 |
| HLA-DPB1 | 33,075,990-33,089,696 | NM_002121.6 | NP_002112.3 |
| MICA | 31,400,711-31,415,315 | NM_000247.3 | NP_000238.1 |
| | | NM_001177519.3 | NP_001170990.1 |
| | | NM_001289152.2 | NP_001276081.1 |
| | | NM_001289153.2 | NP_001276082.1 |
| | | NM_001289154.2 | NP_001276083.1 |
| MICB | 31,494,918-31,511,124 | NM_005931.5 | NP_005922.2 |
| | | NM_001289160.2 | NP_001276089.1 |
| | | NM_001289161.2 | NP_001276090.1 |

*the nucleotide positions correspond to those in Accession No. NC_000006.12 of the GENBANK ® biosequence database.
**denotes that the sequence is derived from the complement of the stated nucleotides in GENBANK ® Accession No. NC_000006.12

In determining an HLA status of cells, tissues, organs, and/or subjects, any screening methodology can be employed. However, it can be advantageous to have an HLA status determined quickly. Thus, in some embodiments the methods of the presently disclosed subject matter relate to determining an HLA status using a polymerase chain reaction (PCR) based method. In methods that employ PCR, amplification primers are designed such that each of the HLA loci sought to be assayed are distinguishable from each other, in some embodiments by the size of the amplification product produced.

Additionally, in some embodiments PCR primers are designed based on sequences that are unique among the HLA nucleic acids that are being screened. By way of example and not limitation, the 3′ untranslated regions of various HLA loci include unique sequences, and PCR primers that distinguish different HLA transcription products can be designed.

In some embodiments, an HLA status of cells, tissues, organs, and/or subjects are determined for one or more of HLA-A, HLA-B, HLA-C, HLA-DRB1/3/4/5, HLA-DQA1, and HLA-DQB1. In some embodiments, an HLA status of cells, tissues, organs, and/or subjects are determined for each of at least HLA-A, HLA-B, HLA-C, HLA-DRB1/3/4/5, HLA-DQA1, and HLA-DQB1. The following Table includes non-limiting examples of amplification primer pairs that can be employed to identify the presence of as well as distinguish among the listed HLA loci:

| Locus | Forward Primer | Reverse Primer |
|---|---|---|
| | Sequence (5′-3′) | Sequence (5′-3′) |
| HLA-A | CCAGACGCCGAGGA | ACACAAGGCAGCTGTC |
| | TGGCC | TCACA |
| | (SEQ ID NO: 1) | (SEQ ID NO: 2) |
| HLA-B | AATCTCCTCAGACGC | CAAGCTGTGAGAGACA |
| | CGA | CATCAG |
| | (SEQ ID NO: 3) | (SEQ ID NO: 4) |
| HLA-C | GCCGAGATGCGGGTCA | ACACAGGCAGCTGTCT |
| | TGG | CAGG |
| | (SEQ ID NO: 5) | (SEQ ID NO: 6) |
| HLA- | CCCCTGGTCCTGTCCTG | CAAGGAAGAGCAGGCC |
| DRB1/ | (SEQ ID NO: 7) | CAGCA |
| 3/4/5 | | (SEQ ID NO: 8) |
| HLA-DPA1 | ATGCGCCCRGAAGACAG | CCCCTGGGCCCGGGG |
| | AATGT | (SEQ ID NO: 10) |
| | (SEQ ID NO: 9) | |
| HLA-DPB1 | CCTTCTTTTCCTGACT | TGYTAACRAAACACAGC |
| | GCAG | AAATGC |
| | (SEQ ID NO: 11) | (SEQ ID NO: 12) |
| HLA-DQA1 | ATGATCCTAAACAAAG | GGTGTCTGGAAGCACCA |
| | CTCTG | ACTG |
| | (SEQ ID NO: 13) | (SEQ ID NO: 14) |
| HLA-DQB1 | GAARAAGKCTTTGCGG | AGTCTCAGGAGTCAGTG |
| | ATCCC | CAG |
| | (SEQ ID NO: 15) | (SEQ ID NO: 16) |

NOTE:
regarding the nucleotides set forth herein above, A is adenine, C is cytosine, G is guanine, T is thymine, K is guanine or thymine, R is guanine or adenine, and Y is thymine or cytosine.

In some embodiments, the amplifying comprises an enrichment step and an amplification step, wherein the amplification step is performed subsequent to the enrichment step; and the amplification employs HLA-specific primers that comprise unique molecular identifiers (UMIs) such that the HLA-specific cDNAs are each uniquely coded. Details with respect to the enrichment step and the use of UMIs is provided herein below in the EXAMPLES.

After the enrichment and amplification steps, the presence of absence of various HLA alleles in the cells, tissues, organs, and/or subjects is determined by analyzing the amplification products produced with the presently disclosed methods. In some embodiments, the amplification products are separated on one or more separation media and the sizes thereof, which are indicative of the presence of particular HLA alleles, are determined.

In some embodiments, however, the various amplification products are determined using flow cells, which in some embodiments can be flow cells that are commercially available from Oxford Nanopore Technologies (Oxford, United Kingdom). Oxford Nanopore Technologies produces several different flow cell platforms that can be employed in the methods of the presently disclosed subject matter, including but not limited to R10.3 Oxford nanopore flow cell, the R10.4 Oxford nanopore flow cell, the R9.4 Oxford nanopore flow cell, and the Oxford nanopore Flongle flow cell. Additionally, Oxford Nanopore Technologies provides various flow cell sequencers that can be employed in the presently disclosed methods. Exemplary such sequencers are the MinION Flow Cell sequencers, optionally the MinION R10.3 Flow Cell sequencer, the MinION Mk1C sequencer, and the MinION Mk1D sequencer; the Promethion sequencer, the Gridion sequencer, the SmidgION sequencer, and the Plongle sequencer, each of which can be deployed consistent with the instructions provided by Oxford Nanopore Technologies.

In some embodiments, the cell, tissue, or organ is a donor cell, tissue, and/or organ for which HLA status is desired is meant for transfer to a recipient or is derived from a donor cell, tissue, or organ meant for transfer to a recipient. As such, in some embodiments the presently disclosed subject matter relates to methods for genotyping donor cells, tissues, and/or organs meant for introduction and/or other administration to a recipient. In some embodiments, the presently disclosed methods comprise, consist essentially of, or consist of (a) providing a donor cell, tissue, and/or organ meant for transplantation into a recipient that comprises mRNA derived from an HLA gene product; (b) reverse transcribing the mRNA to produce a pool of cDNAs, wherein the pool of cDNAs comprises a plurality of HLA-specific cDNAs; (c) amplifying the cDNAs that result from reverse transcription of HLA-specific mRNAs to produce a pool of HLA-specific cDNAs, wherein (i) the HLA-specific mRNAs are transcription products of one or more of HLA-A, HLA-B, HLA-C, HLA-E, HLA-F, HLA-G, HLA-H, HLA-DRB1, HLA-DRB3, HLA-DRB4, HLA-DRB5, HLA-DQA1, HLA-DQB1, HLA-DPA1, HLA-DPB1, MICA, and MICB loci, optionally for each of at least HLA-A, HLA-B, HLA-C, HLA-DRB1/3/4/5, HLA-DQA1, and HLA-DQB1; (ii) the amplifying comprises an enrichment step and an amplification step, wherein the amplification step is performed subsequent to the enrichment step; and (iii) the amplification employs HLA-specific primers that comprise unique molecular identifiers (UMIs) such that the HLA-specific cDNAs are each uniquely coded; and (d) determining the sequence of each HLA-specific cDNAs, whereby the donor cell, tissue, and/or organ meant for transplantation into a recipient is genotyped. In some embodiments, the donor cell, tissue, and/or organ is from a living donor and in some embodiments the donor cell, tissue, and/or organ a cadaveric cell, tissue, and/or organ.

In some embodiments, the amplifying identifies each of HLA-A, HLA-B, HLA-C, HLA-E, HLA-F, HLA-G, HLA-H, HLA-DRB1, HLA-DRB3, HLA-DRB4, HLA-DRB5, HLA-DQA1, HLA-DQB1, HLA-DPA1, HLA-DPB1, MICA, and MICB loci if present in the sample, but any informative subset of these loci can also be identified, as desired. By way of example and not limitation, in some embodiments each of the HLA-A, HLA-B, HLA-C, HLA-DRB1/3/4/5, HLA-DQA1, and HLA-DQB1 loci are identified if present in the sample. In such an embodiment, the HLA-specific primers can comprise HLA-specific sequences, which in some embodiments can comprise, consist essentially of, or consist of the following pairs of oligonucleotide sequences:
(a) For HLA-A: SEQ ID NO: 1 and SEQ ID NO: 2;
(b) For HLA-B: SEQ ID NO: 3 and SEQ ID NO: 4;
(c) For HLA-C: SEQ ID NO: 5 and SEQ ID NO: 6;
d For HLA-DRB1/3/4/5: SEQ ID NO: 7 and SEQ ID NO: 8;
e For HLA-DPA1: SEQ ID NO: 9 and SEQ ID NO: 10;
f For HLA-DPB1: SEQ ID NO: 11 and SEQ ID NO: 12;
g For HLA-DQA1: SEQ ID NO: 13 and SEQ ID NO: 14; and
h For HLA-DQB1: SEQ ID NO: 15 and SEQ ID NO: 16.

As with other methods of the presently disclosed subject matter, the presence or absence of particular HLA loci can be determine using an Oxford Nanopore Technologies (ONT) flow cell, optionally an R10.3 flow cell, an R10.4 flow cell, an R9.4 flow cell, and a Flongle flow cell. ONT flow cells can be analyzed using flow cell sequences also available from Oxford Nanopore Technologies. Exemplary such ONT flow cell sequencers include the ONT MinION R10.3 Flow Cell sequencers, including MinION sequencers, MinION Mk1C sequencers, Promethion sequencers. Gridion sequencers, MinION Mk1D sequencers, SmidgION sequencers, and Plongle sequencers.

As described herein, in some embodiments of the presently disclosed methods, an HLA status is determined based on analysis of nucleic acids present in biological samples, which can optionally be biological samples isolated from subjects (e.g., donors or recipients). Any biological sample that contains the nucleic acids to be analyzed can be employed. Exemplary samples include blood samples, biopsy samples, cell scrapings, etc. In some embodiments the sample is isolated from a donor, and in some embodiments the donor is deceased (e.g., the donor is an organ donor).

With respect to the PCR steps particularly disclosed in the EXAMPLES, it is noted that in each case different PCR conditions could be employed to accomplish the same goals as those recited. By way of example and not limitation, the enrichment steps can be in some embodiments 10 cycles, but indeed less than or more than 10 cycles can be employed, including but not limited to 5, 6, 7, 8, 9, 10, 11, 12, 13, 14, 15, 16, 17, 18, 19, 20, or more than 20 cycles. Similarly, in some embodiments attachment of UMIs employ 2 cycles, and the final PCR steps include in some embodiments 25 cycles. However, particularly with respect to the final PCR steps, a number of PCR cycles other than 25 can be employed. In some embodiments, a PCR-based reaction strategy of the presently disclosed subject matter can include final amplification using in some embodiments 15, 16, 17, 18, 19, 20, 21, 22, 23, 24, 25, 26, 27, 28, 29, 30, or more than 30 cycles.

Also similarly, depending on the particular nucleotide sequences of the primers employed, different annealing temperatures can be employed in order to achieve optimal amplification conditions.

Methods for determining optimal PCR amplification conditions are known in the art, the numbers of cycles and the various temperatures at which each part of a cycle is employed, the length of time for each part of a cycle, and the input amounts/masses and/or volumes of various reagents and nucleic acids to be amplified can be modified as needed to achieve optimal amplification.

Furthermore, it is noted that in some embodiments, the PCR reactions are multiplexed such that a single PCR reaction can be employed to identify a plurality of HLA genotypes simultaneously. It is recognized, however, that whereas in some embodiments each of the HLA-A, HLA-B, HLA-C, HLA-DRB1/3/4/5, HLA-DQA1, and HLA-DQB1 loci can identified if present in the sample simultaneous, each of these HLA loci can be interrogated in individual PCR reactions. With respect to PCR reactions that are multiplexed, the PCR primers employed can be designed such that the interrogated loci will produce a PCR amplification products of different sizes than each other, thereby allowing a single medium (e.g., a single lane of a gel) to be employed for genotyping a sample with respect to multiple loci.

### EXAMPLES

The following EXAMPLES provide illustrative embodiments. In light of the present disclosure and the general level of skill in the art, those of skill will appreciate that the following EXAMPLES are intended to be exemplary only and that numerous changes, modifications, and alterations can be employed without departing from the scope of the presently disclosed subject matter.

### Materials and Methods for the EXAMPLES

Samples. Eighteen unique samples were collected in acid citrate dextrose (ACD) blood tubes from patients undergoing evaluation for solid organ transplantation and healthy donors. Patient and donor demographics are characterized in Table 1. Table 1 describes the patient cohort age, sex, and race distribution. One patient did not identify with any race and is referred to as race Unknown in Table 1.

DNA extraction and quantitation. Genomic DNA (gDNA) was isolated from peripheral blood using MAXWELL®, RSC Whole blood DNA kits (Promega Corporation, Madison, Wisconsin, United States of America) according to the manufacturer's instructions. Briefly, 500 μL of the buffy coat or whole blood was added to the RSC extraction cartridge, and 75 μL of nuclease-free water (included in the kit) was added to the elution tube. The cartridge was loaded onto the Maxwell RSC, resulting in isolated gDNA that was ready for downstream applications. Genomic DNA did not undergo any additional purification and was quantitated using the QUANTUS™ Fluorometer and QUANTIFLUOR® ONE dsDNA system (Promega).

RNA extraction and purification. Total RNA was isolated from peripheral blood using a MAXWELL® RSC SimplyRNA blood kit (Promega) according to the manufacturer's instructions. Briefly, 2.5 mL of whole blood was combined with 7.5 mL of cell lysis solution and then incubated for 10 minutes at room temperature. During this process, the red blood cells were lysed and the white blood cells remained intact. The solution was spun for 10 minutes at 3,000×g and the supernatant was discarded. 200 μL of chilled 1-thioglycerol/homogenization solution, 200 μL of lysis buffer, and 25 μL of Proteinase K were added to the cell pellet, mixed, and incubated at room temperature for 10 minutes. 10 μL of DNase I solution was added to the RSC extraction cartridge in one of the chambers, and the cell lysate was added to another. 50 μL of nuclease-free water (included in the kit) was added to the elution tube. The resulting total RNA was used for downstream applications.

Some RNA samples underwent additional purification using the RELIAPREP™ RNA Cleanup and Concentration System (Promega) according to the manufacturer's instructions. Membrane binding solution was added to the RNA at half the volume of the RNA (12.5 μL), and 100% isopropanol was added at one and a half times the volume of the RNA (37.5 μL). A series of washes and centrifugations were performed with the provided column wash solution and RNA wash solution, resulting in a final elution in 15 μL with nuclease-free water.

Measurement of RNA quality and quantitation. RNA fragmentation and quantitation were determined using TapeStation 4200 RNA ScreenTape Assay (Agilent Technologies Inc., Santa Clara, California, United States of America) following the manufacturer's instructions. Briefly, 1 μL of RNA was added to 5 μL of sample buffer and loaded into the TapeStation. The TapeStation plots the RNA fragment size (nt) against the sample intensity (FU) and generates an RNA integrity number (RIN). The TapeStation RNA ScreenTape Assay separates, images, and analyzes RNA from 50-6000 nt. According to the manufacturer, the assay has a quantitative range of 25-500 ng/μL and a quantitative precision of 10% CV. The RIN functional range was 25-500 ng/μL. The RIN algorithm considers the peak height of the 18S peak to the background signal to calculate the RIN value for total RNA. RIN is on a scale of 1 to 10 where 10 represents highly intact RNA and a low RIN indicates a strongly degraded RNA sample.

cDNA synthesis. cDNA synthesis was performed using the SUPERSCRIPT™ IV (SSIV) First-Strand Synthesis System (Thermo Fisher Scientific, Inc., Waltham, Massachusetts, United States of America). 100 ng of total RNA was combined with 1 μL of 10 mM dNTP and 1 μL of 50 μM Oligo d (T)₂₀ primer and incubated for 5 minutes at 65° C. and then placed on ice. A reaction tube was made consisting of 4 μL 5× SSIV buffer, 1 μL 10 mM DTT, 1 μL Ribonuclease Inhibitor, and 1 μL SUPERSCRIPT™ IV Reverse Transcriptase resulting in reverse transcription (RT) Reaction Mix. The RT reaction mix was added to anneal RNA and then incubated at 53° C. for 10 minutes followed by 80° C. for 10 minutes. This resulted in cDNA being used in downstream applications.

HLA Unique Molecular Identifier (UMI) Assay. A series of PCRs and purifications were utilized to achieve the final enriched PCR product which was an HLA-locus specific UMI amplicon. 1 μL of cDNA was combined with 7.5 μL 2× PLATINUM™ SuperFi II Green PCR master mix (Thermo Fisher Scientific), 0.6 μL each of HLA gene-specific forward and reverse UMI tagged primers (see Tables 2 and 3) at a concentration of 10 μM (Bettinotti et al., 2003; Lank et al., 2012), 0.5 μL 25 mM MgCl₂ (Thermo Fisher Scientific) and 4.9 μL of nuclease-free water (Ambion Inc., Austin, Texas, United States of America). Each sample had an amplification for HLA-A, HLA-B, HLA-C, HLA-DRB1/3/4/5, HLA-DQA1, HLA-DQB1, HLA-DPA1, and HLA-DPB1, generating 8 amplicons per sample. A PCR was performed with an initial denaturation at 98° C. for 3 minutes, followed by 2 cycles of denaturation at 98° C. for 30 seconds, annealing with a touchdown from 66° C. to 60° C. with a ramp rate of 0.2° C./second holding at 60° C. for 60 seconds, and extension at 72° C. for 90 seconds, with a final extension of 72° C. for 5 minutes. The first PCR was followed by an enzymatic removal of unincorporated gene-specific UMI primers by adding 1.5 μL of nuclease-free water and 0.75 μL each of thermolabile exonuclease I (New England Biolabs, Ipswich, Massachusetts, United States of America) and Quick calf intestinal phosphatase (New England Biolabs) and then incubated at 37° C. for 15 minutes followed by enzyme inactivation at 80° C. for 2 minutes. To reduce off-target amplification, cleanup was performed using a 0.6× ratio of solid-phase reversible immobilization (SPRI) Select beads (Beckman Coulter, Brea, CA) and two washes with 80% EtOH. Each amplicon was eluted off the beads with 18 μL of nuclease-free water (Ambion Inc.). The eluate was used in the second round of PCR where it was combined with 25 μL of 2× PLATINUM™ SuperFi II Green PCR master mix, 0.5 μL each of 10 μM UVP forward and reverse primers (Resource Centre, 2017), 2 μL 25 mM MgCl₂ and 4 μL of nuclease-free water. Thermal cycler conditions were initial denaturation at 98° C. for 3 minutes, followed by 5 cycles of denaturation at 98° C. for 30 seconds, annealing with a touchdown from 70° C. to 63° C. with a ramp rate of 0.2° C./second holding at 63° C. for 10 seconds, extension at 72° C. for 90 seconds, 5 cycles of denaturation at 98° C. for 20 seconds with an extension of 72° C. for 2 minutes and a final extension at 72° C. for 5 minutes. Another round of SPRI bead cleanup using a 0.6× ratio and two washes with 80% EtOH was performed. The final round of PCR was performed using the eluate and 25 μL of 2× PLATINUM™ SuperFi II Green PCR master mix, 0.5 μL each of 10 μM UVP forward and reverse primers, which was the same master mix makeup that was used from the second round of amplification. The PCR conditions were initial denaturation at 98° C. for 3 minutes, 25 cycles of denaturation at 98° C. for 20 seconds with an extension at 72° C. for 90 seconds, and a final extension at 72° C. for 2 minutes. A final SPRI bead cleanup, as previously described, was performed using 80% EtOH washes.

Quantification and dilution of amplicons. Each PCR product was analyzed with the QUBIT® 2.0 fluorometer (Thermo Fisher Scientific, Inc., Waltham, Massachusetts, United States of America) dsDNA broad range (BR) assay. A portion of each PCR product was diluted with nuclease-free water to an estimated concentration of 20 ng/μL in 20 μL. A subsequent QUBIT® analysis was performed with the dsDNA BR assay to assure accurate concentrations into the library preparation.

Library Preparation. HLA gene-specific amplicons for one sample (8 HLA loci) were combined into one tube in an equal molar ratio based on their femtomolar (fmol) concentration. A total of 350-400 fmol was targeted for each sample, and the volume was brought to 48 μL with nuclease-free water. Libraries were prepared according to the Native Barcoding Amplicons protocol using the Ligation Sequencing kit (SQK-LSK109) and Native Barcoding Expansion Kits (EXP-NBD104 and EXP-NBD114) from Oxford Nanopore Technologies (Oxford, United Kingdom) as follows. For a given sample, the HLA loci were pooled on an equimolar basis. In a PCR tube, 48 μL of pooled amplicon DNA, 3.5 μL of NEBNEXT® FFPE DNA Repair Buffer (New England Biolabs), 2 μL of NEBNEXT® FFPE DNA Repair Mix (New England Biolabs), 3.5 μL of Ultra II End-prep reaction buffer (New England Biolabs) and 3 μL Ultra II End-prep enzyme mix (New England Biolabs) were combined. Samples were mixed gently, spun down, and incubated at 20° C. for 5 minutes and then at 65° C. for 5 minutes. A bead-based cleanup was performed using SPRI Select beads at a 1:1 ratio, and two washes were performed using 70% EtOH. Samples were eluted in 25 μL of nuclease-free water and then quantified using the QUBIT® dsDNA high sensitivity (HS) assay (Thermo Fisher Scientific). Each sample was given a unique barcode utilizing the Native Barcoding Expansion Kit. A target of 150-300 fmol in a volume of 22.5 μL for each sample was used, combined with 2.5 μL of native barcode and 25 μL Blunt/TA Ligase Master Mix (New England Biolabs), and was mixed by pipetting and incubated at room temperature for 10 minutes. A bead-based cleanup was performed using SPRI Select beads at a 1:1 ratio (50 μL) and two washes were performed using 70% EtOH. Samples were eluted in 26 μL of nuclease-free water and quantified using the QUBIT® dsDNA HS assay. Equimolar amounts of each barcoded sample were pooled together into a 1.5 mL tube with a target of 350-400 fmol for the pool. The pool was brought up to 65 μL with nuclease-free water, to which 5 μL of Adapter Mix, 20 μL of 5× NEBNEXT® Quick Ligation Reaction Buffer (New England Biolabs), and 10 μL of Quick T4 DNA Ligase (New England Biolabs) were added, mixed gently, and incubated at room temperature for 10 minutes. 50 μL of SPRI Select beads was added to the tube, pipetted, and incubated on a rotator for 5 minutes. Tubes were placed on a magnetic rack for the beads to pellet, and the supernatant was pipetted off. Two washes using 250 μL each of short fragment buffer from the Ligation Sequencing Kit were performed. Final elution was performed using 15 μL of Elution Buffer from the Ligation Sequencing Kit and incubated for 10 minutes at room temperature. The final library was quantified with a QUBIT® dsDNA HS assay. Then, 100 fmol of the final library was used for sequencing. Sequencing was performed using a MinION R10.3 flow cell and loaded on the MinION Mk1C (Oxford Nanopore Technologies) with firmware version MinION FPGA 2.3.2 and MinKNOW version 21.02.2 using the high-accuracy base-calling module. Samples were run individually and in batches containing up to 7 pooled samples.

Reference HLA Genotyping. Isolated gDNA was HLA genotyped using the AlloSeq Tx17 assay (CareDx, Inc., Brisbane, California, United States of America). 10 μL of gDNA with a concentration of at least 10 ng/μL was utilized and samples were prepared following the manufacturer's instructions. First, a tagmentation step is performed on the gDNA, followed immediately by indexing PCR, which barcodes each sample. Double-tailed size selection and purification with SPRI beads (CareDx) were performed to target only the fragments of DNA of appropriate size. Samples were then combined (pooled) with up to 12 samples per pool. Pools were hybridized with the AlloSeq Tx17 probe panel with a slow ramp down from 98° C. to 62° C. ending with incubation at 62° C. for at least 90 minutes and up to overnight. The 17 probes included in the AlloSeq Tx17 are designed to hybridize to the HLA-A, HLA-B, HLA-C, HLA-E, HLA-F, HLA-G, HLA-H, HLA-DRB1, HLA-DRB3, HLA-DRB4, HLA-DRB5, HLA-DQA1, HLA-DQB1, HLA-DPA1, HLA-DPB1, MICA, and MICB loci.

While the AlloSeq Tx17 kit contains probes for all 17 of these loci, the 11 classical HLA loci that are of most interest for comparative analysis. Exemplary oligonucleotide primers for these 11 classical HLA loci were as follows:

A series of stringent washes with suppled buffers were performed post hybridization to capture the targets correctly. This was followed by a short enrichment PCR and post-amplification cleanup per the manufacturer's instructions using provided SPRI beads and 80% EtOH. Libraries were quantitated via QUBIT® t dsDNA BR Assay. Sequencing was performed using the MiSeq platform (Illumina, Inc., San Diego, California, United States of America) with libraries denatured and diluted to 20 μM, and a 1% concentration of PhiX was added. A MiSeq cartridge with v2 chemistry and a microflow cell were utilized with 2×150 sequencing.

Bioinformatics. All sequencing files for a patient were combined and used to generate consensus sequences based on the minimum number of reads grouped by UMI. Experiments used a minimum read of 1 per UMI (min1) or 10 (min10), which is a UMI group. Additionally, using a UMI group of 1 enabled PCR deduplication. Adapters, primer sequences, and sample barcodes were removed using porechop (Hughes et al., 2020; Wick et al., 2018). UMI grouping was performed using vsearch (Rognes et al., 2016), and consensus sequences were generated using Medaka (version 1.1.3; Xu et al., 2020). Consensus sequences generated were used for subsequent downstream analysis. The consensus sequence generation code is available at https://github.com/nanoporetech/pipeline-umi-amplicon/tree/cdna-no-mapping.

RNA-Seq HLA typing and allele-specific transcript quantitation. HLA typing was performed using a modified version of NGSEngine enabling cDNA as the input sequence (GenDx, Chicago, Illinois, United States of America). See also Liu et al., 2018b; Liu & Berry, 2020. When performing the assay, both pipelines were used to complement each other to achieve the genotyping results that were generated using NSGEngine without manual adjustments. The UMI count for each HLA allele was generated from the number of consensus reads, each associated with a unique UMI, which aligned to an allele using the Athlon2 pipeline (Liu. 2021). The alignment was implemented using minimap2 version 2.17 (Li, 2018) and cDNA sequences from the IPD-IMGT/HLA database v3.38. The accuracy of consensus reads from different UMI group sizes (i.e., min1 versus min10) was calculated as a percentage of the number of matching bases over the length of the alignment region.

Normalization of unique molecular identifier counts and mRNA transcripts. To normalize UMI counts across patients, the method described in Johansson et al., 2021 was used. Briefly, the sum of each HLA gene-specific UMI count was divided by the allele-specific UMI count, multiplied by the total UMI count for the patient and scaled to 1 million reads. The output was then transformed using log base 2.

Statistical Analysis. Statistical analysis was performed using R (version 4.1.1) and GraphPad Prism (version 9.3.1). One-way analysis of variance using Geisser-Greenhouse correction, followed by the Tukey multiple-comparisons test. Linear regression was used to evaluate correlation with R² values derived from the Pearson correlation coefficient. The Kruskal-Wallis test, paired t test, independent t test, and Mann-Whitney U t test were utilized to determine statistical significance as appropriate. P values that were <0.05 were considered statistically significant.

### Introduction to the EXAMPLES

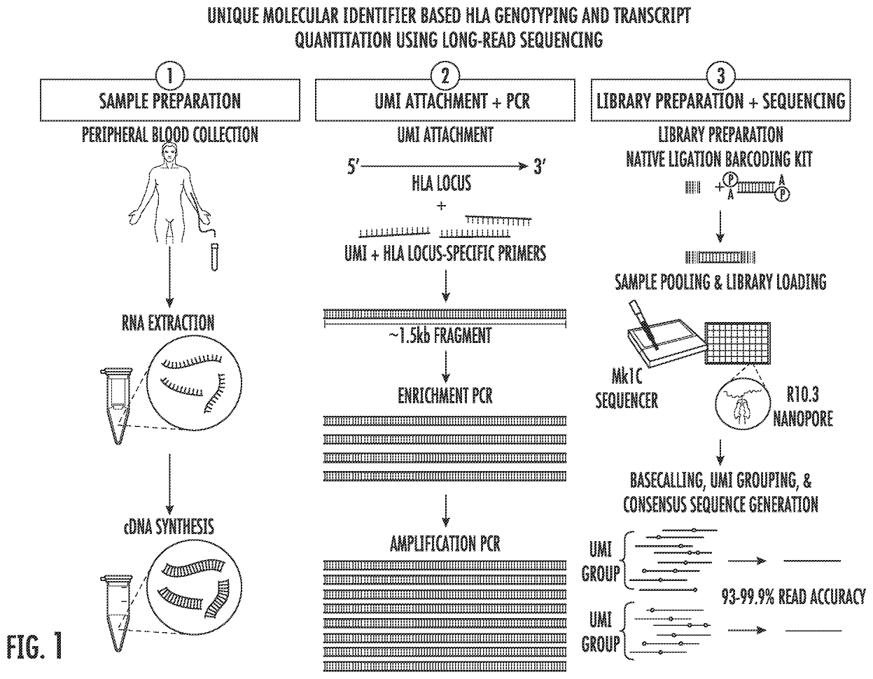

An objective was to further evaluate whether long-read RNA sequencing could be utilized for rapid HLA genotyping and HLA RNA transcript expression. The overall assay schematic is shown in FIG. 1. In short, total RNA was isolated from peripheral blood and a portion of the total RNA was purified. RNA fragmentation and quantitation were determined and an RNA integrity number (RIN) was calculated for each sample based on RNA fragment size and sample band intensity. The RIN value is on a scale of 1 to 10, where 10 represents highly intact RNA and a low RIN indicates a more fragmented RNA sample. cDNA synthesis was then performed after which PCR amplification and purification were utilized to achieve the final enriched HLA-locus specific UMI tagged amplicon. The HLA gene-specific amplicons for each patient were then used for library preparation and barcoding for use on the Oxford Nanopore Technologies (ONT) platform. The final library with a target of 100 fmol was used for sequencing which was performed using the MinION R10.3 Flow Cell and loaded on the MinION Mk1C using the high-accuracy base calling module.

In the cohort of 18 patients, the count of different alleles was as follows: 12 (HLA-A), 17 (HLA-B), 12 (HLA-C), 3 (HLA-DPA1), 12 (HLA-DPB1), 5 (HLA-DQA1), 5 (HLA-DQB1), 11 (HLA-DRB1), 3 (HLA-DRB3), 1 (HLA-DRB4), and 1 (HLA-DRB5). The patient median age was 53 years old, with ages ranging from 25 to 67 years of age. Of the patients, 61.1% were female (see Table 1). The cohort included patients of African American and European American ethnicities with the majority of the patients being of African American ethnicity or descent (61.1%).

### Example 1

Metrics for cDNA-Based HLA Typing on the ONT Platform

To better assess the quality of RNA samples obtained from the patient samples, isolated RNA from each patient was measured by the TapeStation 4200 (Agilent), and an RNA integrity number (RIN) was calculated from the band density and sample fragmentation (FIG. 1). The patient samples displayed a heterogeneous distribution of RIN values ranging from 3.0 to 8.1, with a median RIN value of 4.9.

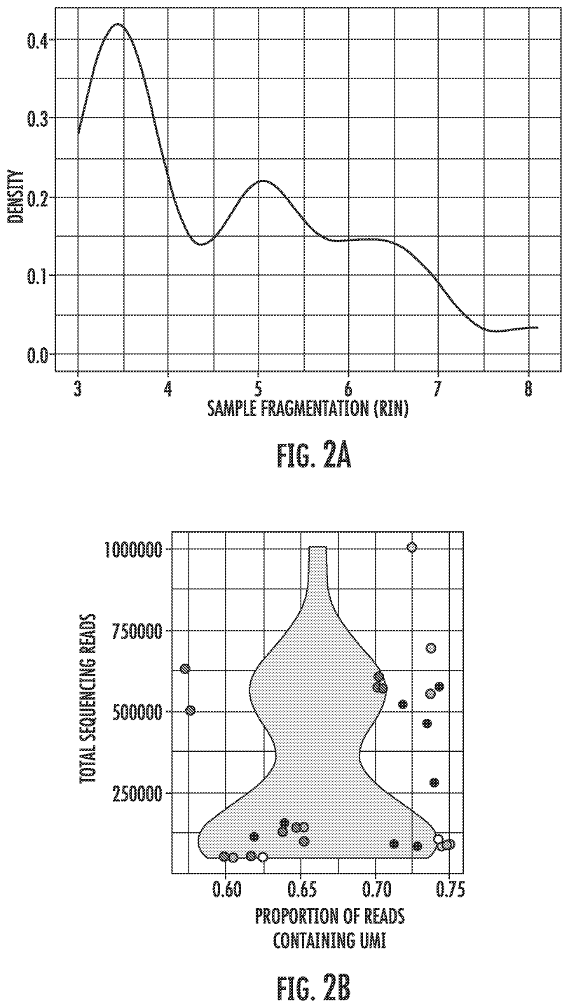

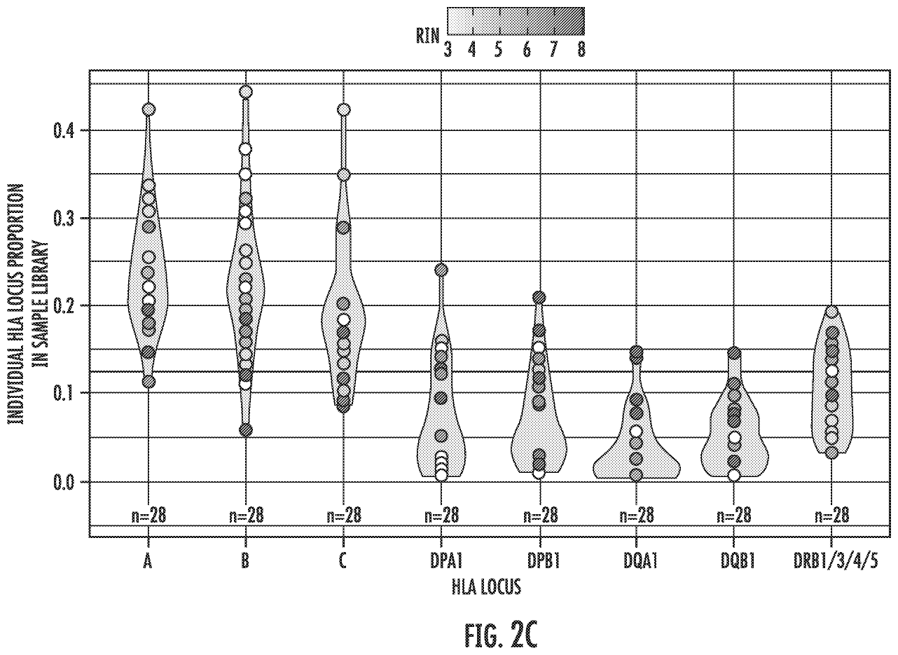

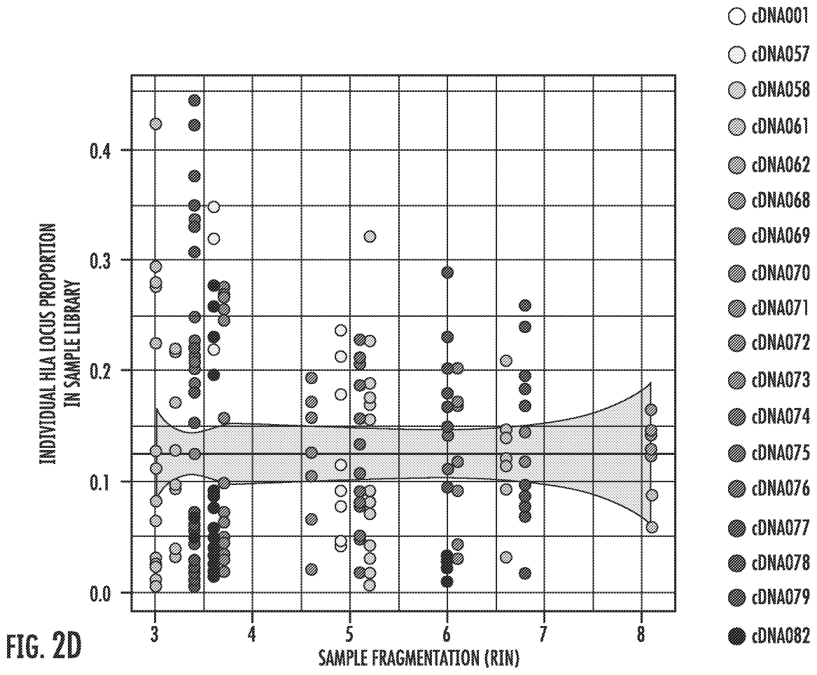

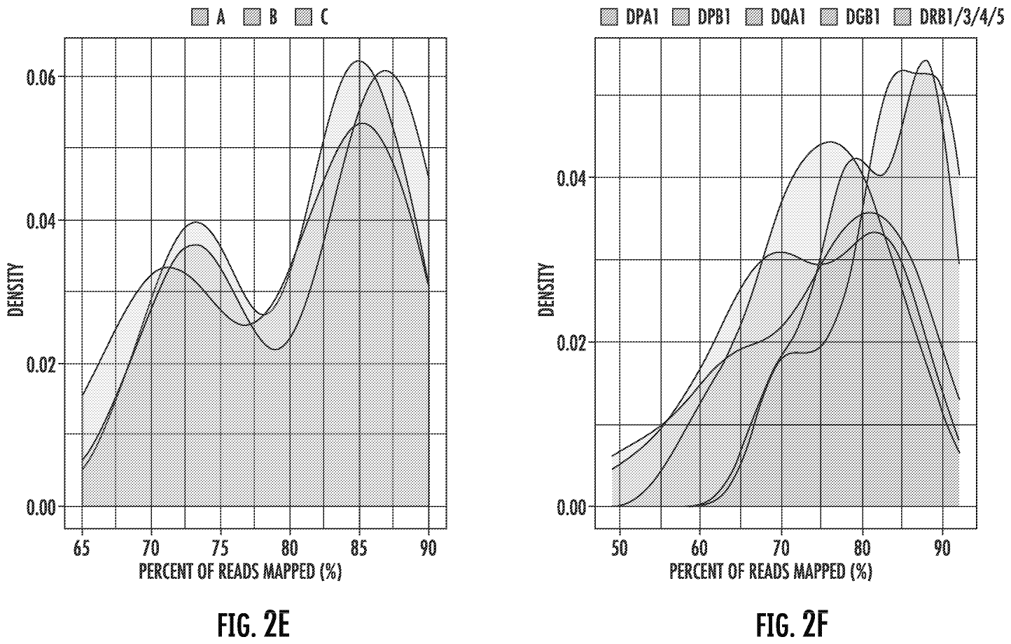

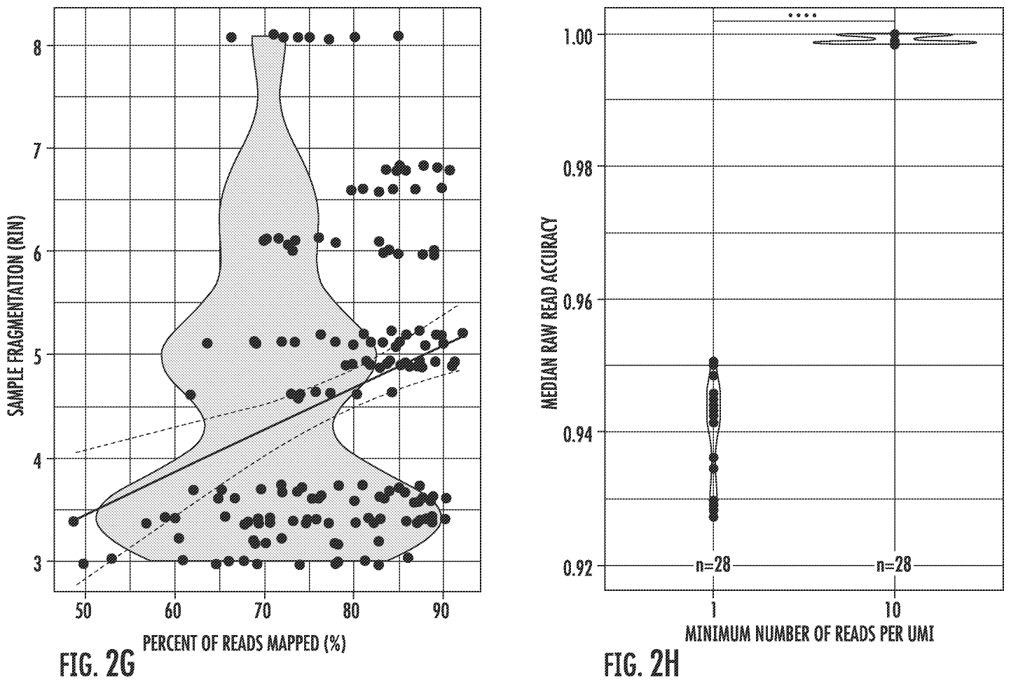

The efficiency of UMI tagging in the assay was also assessed. To this end the total reads for each patient were quantified and the proportion of those reads that contained a UMI was determined (FIG. 2B). Samples with higher total reads tended to have a higher proportion of UMI. More importantly, all patient sample sequences demonstrated that the majority of reads acquired were UMI labeled with the proportion of reads that were UMI tagged ranging from 57.2% to 75.0% (FIG. 2B).

To investigate and assess the collection of reads obtained from the classical HLA loci, the proportion of reads mapped to each of the classical HLA loci of the total reads sequenced was investigated (FIG. 2C). Most samples exhibited the highest proportion of reads from the class I HLA loci, HLA-A, -B, and -C, with class II HLA loci displaying a much lower proportion of total reads on average. To determine if sample RIN influenced the proportion of reads obtained across the classical HLA loci, the distribution of reads across the HLA loci was compared with the RIN value for each patient sample (FIG. 2D). The data are heterogeneous. Nearly all samples, those with lower and higher RIN values, shared similar distributions of sequencing reads across the HLA loci, with a higher proportion of class I HLA loci compared to class II HLA loci.

To further evaluate whether relative amounts of RNA input might influence the alignment ability of the assay, the TapeStation band density as measured by sample fluorescent intensity was compared to the percentage of reads that were UMI identified and able to be mapped to HLA loci. All of the HLA loci demonstrated a similar trend with samples that had a higher band density also displaying a greater proportion of reads that were able to be aligned for class I (FIG. 2E) and class II (FIG. 2F). Since, the band density demonstrated this trend, whether sample fragmentation influenced the mapping of reads to a specific HLA loci was ascertained. To do this, the percentage of reads that were aligned to HLA loci was investigated and compared those values to the patient sample RIN value (FIG. 2G). The samples showed a wide range of reads that could be mapped to HLA loci ranging from 49% to 92%. While the R² value derived from comparing the RIN value to the percentage of UMI tagged reads that are mapped to HLA loci is rather poor (Pearson r, R²=0.075), there does appear to be a trend that is noticeable when examining the results (Pearson r, P value=0.0001). This comparison demonstrated that there could be a correlation between the fragmentation of samples and the ability of the samples to be successfully aligned to HLA loci. This trend implied that HLA loci from patient samples with a higher RIN result in a greater proportion of reads that can be successfully mapped.

### Example 2

### HLA Genotyping

As described herein above in the Materials and Methods for the EXAMPLES section and similar to other NGS sequencing assays, this assay utilized UMIs to tag unique transcripts to quantity them and generate a consensus sequence for each unique transcript. The consensus sequences were generated by grouping sequences tagged with the same UMI. The number of reads within a specific UMI was used to set the minimum number of reads required to create the consensus sequence. To evaluate the accuracy of different UMI group thresholds, the accuracy of patient HLA typing using a minimum UMI read count of 1 (min1) and a minimum UMI read count of 10 (min10) were evaluated (FIG. 2H). Not unexpectedly, the output consequence accuracy was significantly higher (p<0.0001) for a min10 (98.7%) than for a min1 (95.9%). See FIG. 2H.

To evaluate the typing concordance of the presently disclosed assay, the resultant HLA genotype was compared using the NGSEngine bioinformatics pipeline to each patient's reference typing results (Table 4). 18 unique patient samples, each of which was typed for 8 to 9 HLA loci per patient, provided a total typing of 314 HLA alleles. Overall, the accuracy was 99.68% (313/314), excluding the genotyping of a DRB5 null allele. The only discrepant typing was one at DQA1, with the reference typing being a DQA1*05:05:01 and the assay resulting the allele as a DQA1*01:01:01.

### Example 3

### Expression of RNA Transcripts at Classical HLA Class I and Class II Loci

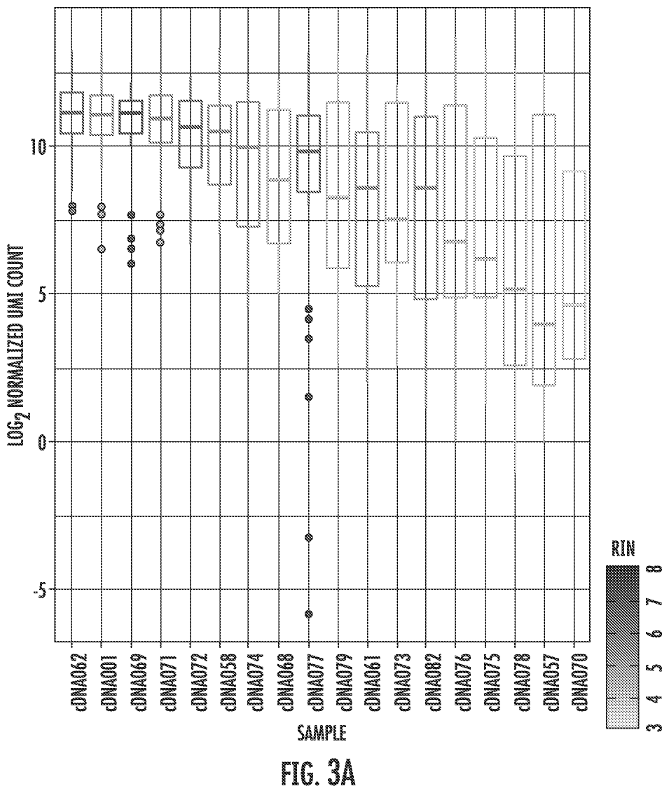

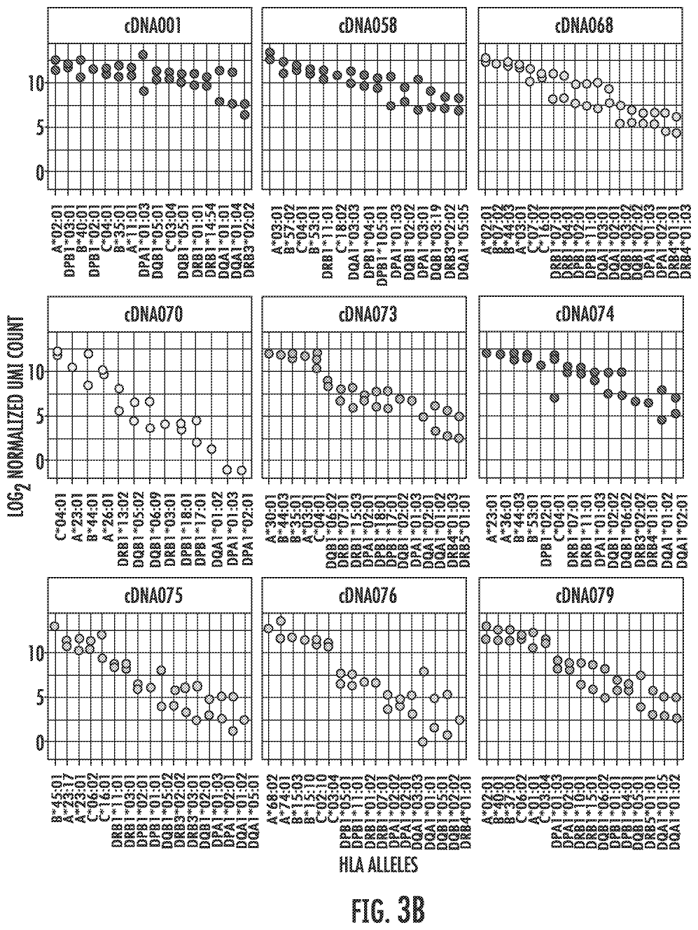

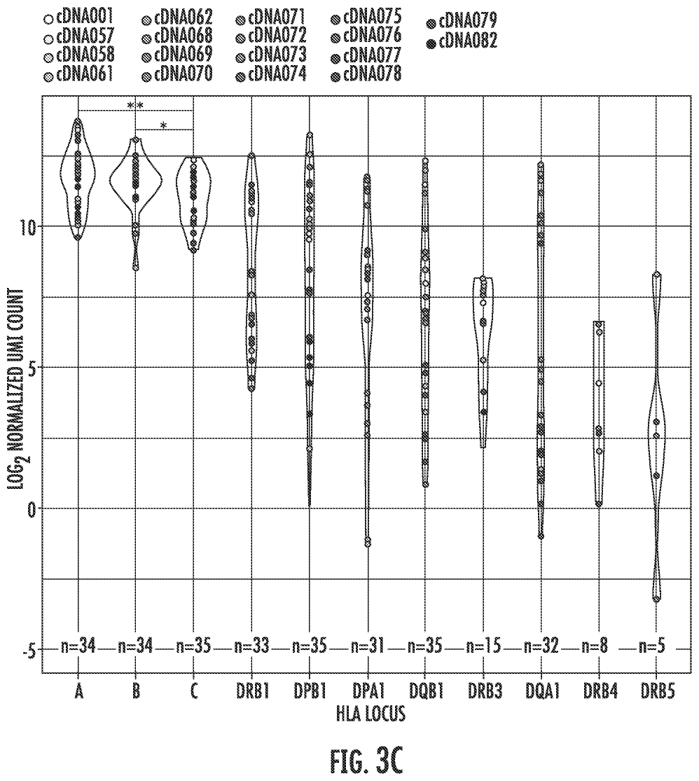

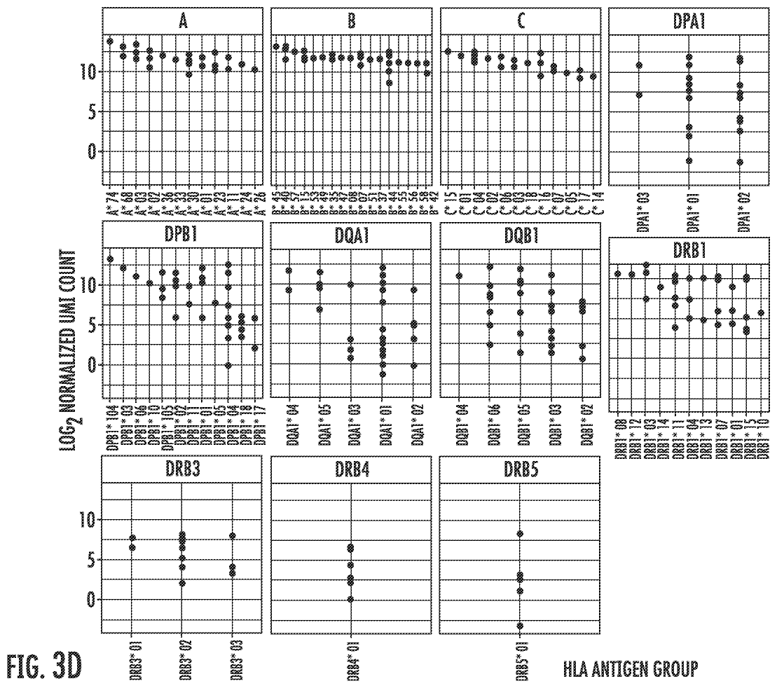

Previous studies have evaluated using RNA-Seq to provide HLA typing. This study evaluated both the ability to provide HLA typing and HLA allele-specific expression using RNA-Seq. The expression of HLA transcripts was determined by counting the UMI-tagged HLA specific reads and described in detail herein above the Materials and Methods for the EXAMPLES section. This method was based on Johansson et al., 2021 and other allele-specific RNA-Seq expression studies. In this way, the UMI count for all patient samples was normalized for each HLA locus and read depth (FIG. 3), the distribution of which was examined for each patient in the presently disclosed cohort (FIG. 3A).

When comparing the expression of transcripts, higher transcript levels were associated with class I transcripts compared to class II transcripts (FIG. 3B). This trend appears to be independent of sample RIN values, as samples with both high RIN and low RIN values exhibited this pattern of expression. FIG. 3B also displays the interassay precision analysis. To this end, 16 of the 18 samples were repeatedly tested using the same input RNA as previously utilized for the primary patient runs. Identical typing results were obtained for the 16 patients, and no discrepancies were noted in the second field of HLA typing resolution. Evaluating the precision comparing the first and subsequent RNA-Seq HLA locus expression results, it was found that they were fairly similar for the class I loci, with the variation being only 7.37%, 6.48%, and 7.28% at the HLA-A, HLA-B, and HLA-C loci, respectively. There was more variation observed in the class II loci, with the lowest coefficient of variation of 24.27% noted for the DRB1 locus. The expression of the other class II loci showed variation ranging from 34.32% at DPB1 to 56.93% at the DQA1 locus.

Of the class II loci, varying degrees of expression were observed. DRB1 alleles demonstrated consistently high levels of transcripts and were significantly higher than DQB1, DQA1, and DRB3/4/5 transcripts (Mann-Whitney U, p value=0.0139, p value=0.0054, and p value<0.0001, respectively), but did not have significantly different transcript levels compared to DPA1 and DPB1 alleles. Of interest, there was some heterogeneity in HLA expression observed among patients in the presently disclosed cohort for class I and class II (FIG. 3C). For example, patient cDNA062 demonstrated higher expression of DPB1, DQB1, and DQA1 transcripts compared to all other loci, including HLA-A, -B, and -DRB1 loci. A patient who exhibited expression patterns that were abnormal from the pattern of expression exhibited by the other patients was that of cDNA071. This patient had DRB1, DPB1, and DPA1 transcript levels that were equally as high as the HLA-A and -B transcripts by UMI count.

The variability of expression between HLA alleles of the different loci (FIG. 3D) was also examined. It was noted that there was greater variance observed in class II loci for the presently disclosed cohort of patients compared to class I loci (Mann-Whitney U, p value=0.0238) with class I loci demonstrating an average variation of 8.22% contrasted by the class II loci which had an average expression variation of nearly 50.59%. Of the class II loci, the DRB1 alleles demonstrated the least amount of variance, 30.12%, and DRB3/4/5, DQB1, and DQA1 exhibited the greatest amount of variance at 59.40/o, 51.51%, and 71.63%, respectively.

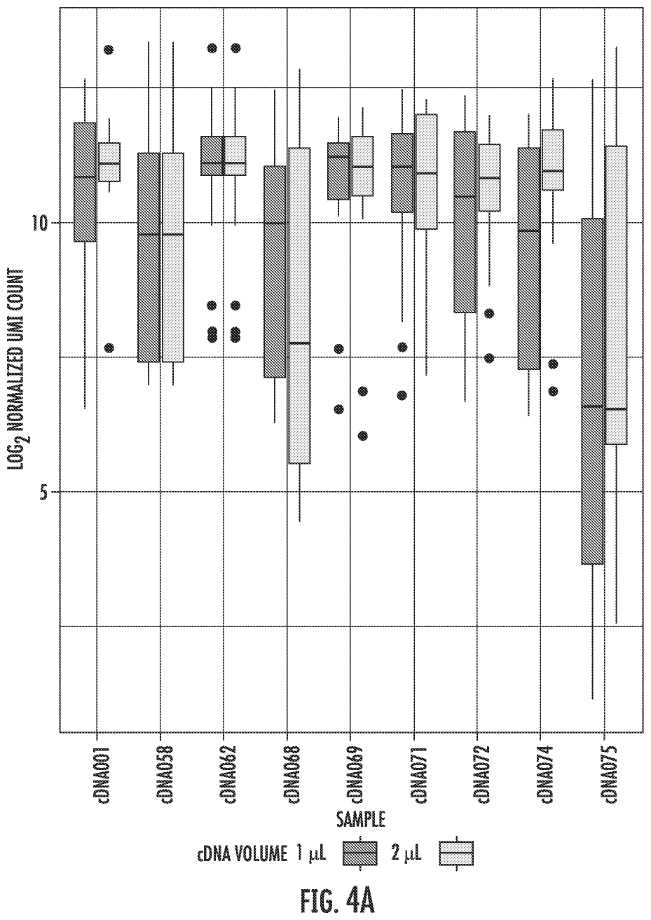

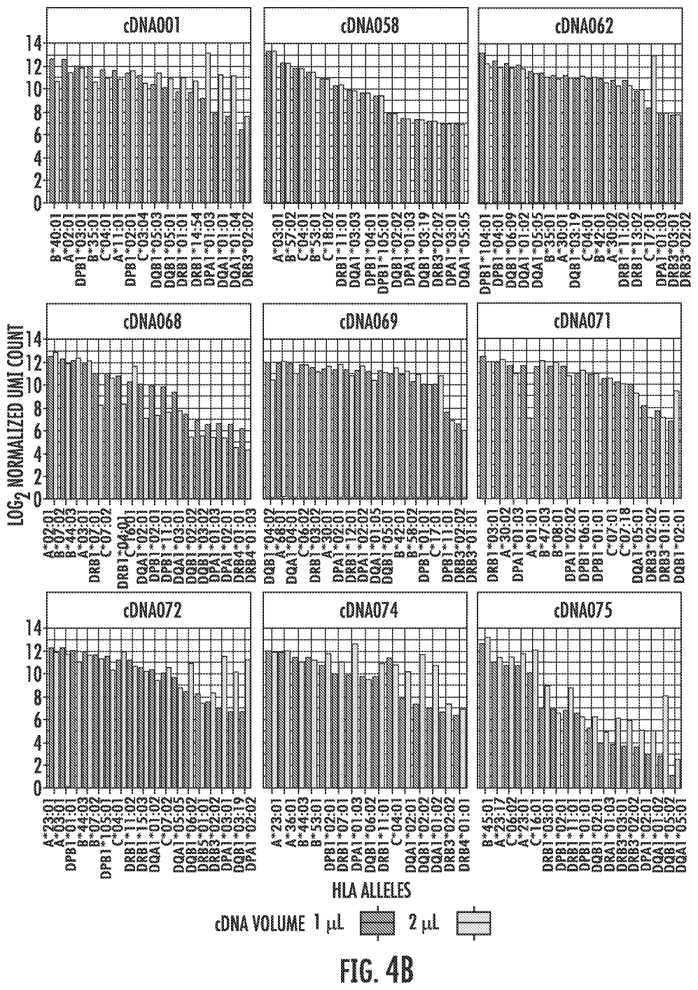

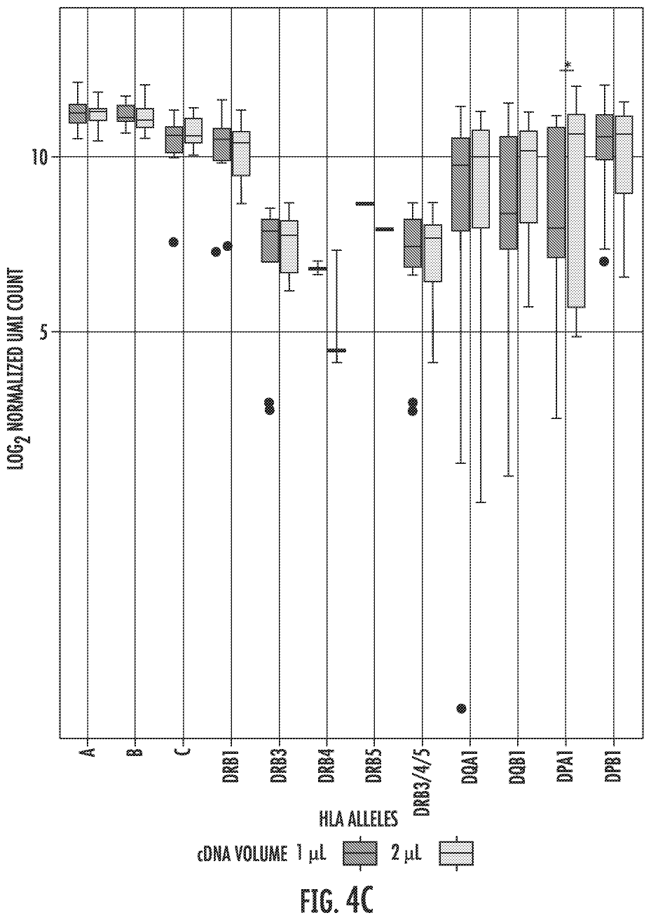

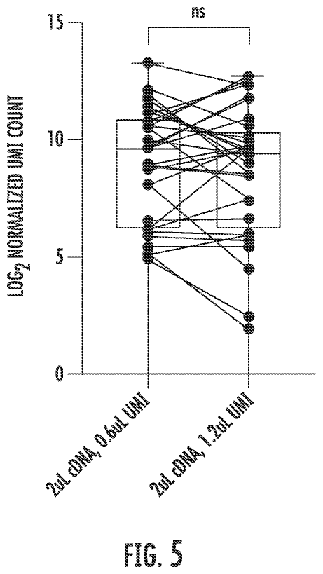

To investigate whether lower input amounts of cDNA might be responsible for the desperate variance observed between class I and class II, 9 of the 18 samples were repeated using twice the volume, doubling the amount of cDNA used initially. While the doubling of input cDNA did increase the UMI count of some of the class II transcripts, the increase was not significantly different for any of class I or class II HLA loci except for the DPA1 locus (FIG. 4). Transcripts at the DPA1 locus where transcript expression significantly increased by 18.96% on average (paired t test, p value=0.034).

### Discussion of the EXAMPLES

The presently disclosed study presents an RNA-Seq assay approach that can provide HLA genotyping and mRNA expression levels of the classical HLA loci as calculated by UMI counting of the HLA loci reads which has been demonstrated to be effective in other studies (Johansson et al., 2021). This method displays excellent accuracy and concordance with the NGS reference typing obtained using Illumina sequencing. The high accuracy is in part a result of setting a minimum threshold to ensure that a high enough number of unique UMIs are utilized to create the allele consensus sequence that is then used to derive the HLA genotyping. The assay had an overall HLA typing accuracy of 99.68% with one discrepant typing. The discrepant typing was a DQA1*05:05:01 that was called as miscalled as a DQA1*01:01:01. Other samples with the same DQA1 allele were typed using the presently disclosed RNA-Seq method. Upon investigation it was concluded that the discrepant typing results were likely related to sample collection as repeat testing did not resolve this particular discrepancy. There was a second typing; however, it was not considered to be discrepant by the presently disclosed methods. This typing was a DRB5*01:08:01N where no transcripts were identified by RNA-Seq, even after repeat testing was performed. While mRNA transcripts of this null allele are likely produced, transcript levels were likely extremely low, possibly due to intracellular degradation as a result of its early transcript termination (Coorter et al., 1997; Barsakis et al., 2019). This is a limitation for any RNA-based HLA typing method compared to a genomic DNA NGS typing method. It is possible that null alleles with highly degraded or no mRNA expression would fail to be typed due to the nature of the typing methodology. This would have low to no clinical impact as null alleles do not demonstrate surface expression on donor tissue. As this is the case, not providing typing for null alleles would not impact clinical practice or patient outcomes in any way. However, the assay does perform well with relatively low transcript levels. Deceased donor typing or smaller batched runs should have less difficulty with low transcript levels as more reads can be acquired, increasing the depth of coverage, which should ameliorate this problem. We also had several samples where one allele portion was extremely low, ranging between 8.1% and 18.9% of all reads aligned to the locus. Eight of the nine (88.9%) samples where this low allele proportion was observed occurred at the DQA1 locus with the remnant one at the HLA-B locus.

As it was observed in the presently disclosed patient cohort and from other studies investigating the expression of HLA loci, DQA1 is one of the classical HLA loci with the lowest amount mRNA expression (Johansson et al., 2021). As a result it is not surprising that this HLA locus resulted in a discrepancy and that this was also the HLA locus that demonstrated the most allelic imbalance for several samples. Additionally, this could be the result of batching many patients per run using this method. If this method were performed for just one or two donors per ONT run, as would normally performed for deceased donor typing by other methods, more reads could be obtained per patient sample and the likelihood of dropout, allelic imbalance, or mistyping as a result of low transcript levels could be mitigated.

Aside from HLA genotyping, this RNA-Seq method also provides HLA allele mRNA transcript expression. Nearly all of the presently disclosed patient samples had a greater amount of UMI counted reads from the class I HLA loci, HLA-A, -B, and -C, with class II HLA loci displaying a lower proportion of total reads on average. These findings are consistent with what would be expected given the difference in proportion of cells in the peripheral blood. Since the presently disclosed RNA was extracted from peripheral blood, there should be more class I mRNA transcripts than class II transcripts given the larger proportion of class I expressing cells compared to class II-expressing cells. The presently disclosed experimental results are similar to those of Johansson et al., 2001, which also utilized an RNA-Seq method to investigate HLA transcript levels. As in their study, it was found that class I loci transcripts were more abundantly expressed than class II transcripts. However, in the presently disclosed cohort of patients, it was determined that of the presently disclosed class I alleles both the HLA-A and -B loci have higher expression than HLA-C. Their study cohort demonstrated lower transcripts of HLA-A, with HLA-C having the highest amount of transcripts of all the HLA loci that they evaluated. This difference is likely due to the population demographic differences between the studies. There is evidence that HLA haplotype is more associated with HLA-C mRNA expression than with allotype or polymorphisms (Bettens et al., 2014). Additionally, it has been demonstrated that age likely influences HLA mRNA transcript levels for HLA class I loci (see Morvan et al., 2001).

This could also be related to the difference in methodology utilized between the studies. The method utilized by Johansson et al. uses a UMI that is attached to the 5′ end of the RNA transcripts using a template-switching oligo (TSO) during first-strand synthesis. This is then followed by amplification of the full-length cDNA. The presently disclosed method performs cDNA synthesis as usual per the manufacturer's instructions and then attaches the UMI to the 5′ end of the transcript after cDNA is synthesized and during PCR enrichment instead. It was found that by attaching the UMI during the PCR enrichment step rather than during cDNA synthesis, it was possible to obtain a higher UMI labeling efficiency. However, this difference in protocol might explain, at least in part, the different class I mRNA expression results observed in the presently disclosed patient samples.

However, the class II findings between this study and that of Johansson et al. are extremely similar. The presently disclosed study provides mRNA expression data for the DRB3 loci. While the expression of mRNA transcripts at the DRB5 locus is fairly heterogeneous, the DRB3 and DRB4 loci demonstrate much less variance of expression. However, for most patients, these transcripts are some of the least abundant measured of HLA loci examined. These results are concordant with studies that have investigated the expression of DRB3/4/5 and compared it to the expression of mRNA transcripts at DRB1, mainly showing that DRB1 transcripts are typically more abundant in lymphocytes than DRB3/4/5 transcripts (Emery et al., 1993; Faner et al., 2010).

The presently disclosed assay did display increased variance between patient samples at the DPB1, DPA1, DQB1, and DQA1 loci. This variance was likely a consequence of two factors. First, it is known that these loci exhibit lower expression than other HLA loci in certain patients and could also be due to allele-specific transcript expression, resulting in higher variance between patient samples. Second, these class II loci transcripts are only expressed by antigen presenting cells, which likely represent the minority of cells from which RNA was harvested. The proportion of these cells in the peripheral blood could also be different between patients, resulting in the high variance observed between patient samples that share the same DP and DQ alleles.

The mRNA expression obtained with this type of assay can contribute immensely in several areas of transplant medicine. Quantification, or even just relative quantitation, of mRNA transcripts for each locus of a patient can assist in improving the evaluation for donor organ rejection based on the immunologic risk profile of the selected recipient. While elevated mRNA transcript levels do not always ensure elevated protein levels, there is an association that most elevated transcript levels result in elevated translation of transcripts into protein. As a result, understanding which transcripts are highly elevated can help predict which proteins would most likely be most prevalent on the surface of donor cells. As an example, in general it might be expected that HLA-C is less expressed on the cell surface and is also typically less immunogenic, however, one of the patients displayed the highest count of mRNA transcripts for one of the HLA-C alleles compared to all other loci. These findings could suggest that the HLA-C loci for this patient might be more immunogenic due to the probably elevated amounts of allelic HLA-C expressed for the donor or that it might be more prudent to identify a matching donor at the HLA-C locus to alleviate potentially more risk at this HLA locus. This same principle could apply to any of the HLA loci where elevated levels of mRNA transcription might suggest an increased immunologic risk for donor recipient pairs.

These assay results are extremely encouraging considering that both highly accurate HLA genotyping and RNA expression were able to be derived from samples that showed both low and high RIN values. Sometimes obtaining samples or utilizing different sample types for DNA or RNA-based assays can be rather challenging. Understanding that this assay can provide reliable results even from highly fragmented RNA samples allows for the possibility that this assay could be used to provide HLA typing and gene expression on alternate sample types where RNA yield is very fragmented, such as in the case of RNA isolation from formalin-fixed paraffin-embedded (FFPE) tissue samples (Levin et al., 2020; Shi et al., 2021). This would allow laboratories to obtain HLA typing and transcript expression from tissue biopsy samples if there were the need to investigate patient physical crossmatch and HLA antibody test results. While the most fragmented samples with RIN values of 3 were able to achieve accurate HLA typing results, it is likely that samples with lower RIN values, exhibiting even more fragmentation, have an increased risk of HLA typing failure.

These results could assist in providing more accuracy and sensitivity for virtual crossmatch determinations (Garcia-Sanchez et al., 2020). By understanding the relative transcript levels or proportion of HLA allele transcript counts, centers would have the ability to use preexisting HLA antibody testing data to better identify which donor antigens are likely to result in a positive physical crossmatch given levels of mRNA expression (Montgomery et al., 2019; Weimer & Newhall, 2019). If a donor has relatively low amounts so mRNA transcripts of a given allele (s) that would otherwise have been assumed to be higher, a negative crossmatch determination could be made with higher confidence. The inverse of this would also be likely true as well, with abnormally increased mRNA transcripts observed, a positive cross match could be predicted with a higher level of confidence. The presently disclosed assay is a tool for enabling a better understanding of how allele-specific HLA transcript expression impacts transplant compatibility and outcomes. More studies are needed to elicit the exact nature of how HLA transcript levels impact immunologic compatibility.

A final advantage to utilizing this assay for clinical typing in the future is the advantage of turnaround times. Using the ONT platform, the assay has a turnaround time of 7 to 8 hours from receipt of the sample in the laboratory to evaluation of final HLA typing results and HLA mRNA expression levels. This is shorter than the time required for current NGS methods used in the clinical transplant medicine laboratory, which normally requires approximately 18-24 hours based on the NGS method utilized. Compared with AlloSeq hybrid capture NGS HLA typing, this assay is quite rapid (FIG. 1), providing complete HLA typing and transcript expression in 6.5 to 7 hours. Compared to the most rapid methods of obtaining deceased donor typing using real-time polymerase chain reaction (RT-PCR) or sequence-specific oligonucleotide (SSO), this assay is slightly more time consuming by several hours. However, the additional information gained, high resolution typing and donor HLA transcript expression would be beneficial and worth the additional assay time. With this RNA-Seq workflow timeline, a deceased donor could be typed and HLA mRNA expression levels could be calculated to provide this additional information to better help transplant centers assess the immunologic risk of a donor-recipient pair. Additionally, donor transcript expression in conjunction with recipient anti-HLA antibody status could be utilized together to provide better virtual crossmatch predictions (Montgomery et al., 2019). The assay could also be utilized for typing living donors. The patients were typed using batches of up to 7 patients. However, the assay has the potential to be scaled up to accommodate a higher number of patient batches, if necessary.

As set forth herein, a novel assay that provides high-resolution HLA typing and allele-specific HLA expression within the time constraints for deceased organ donor allocation is described. While the assay may not provide immediate benefits for the current organ allocation scheme, it would provide additional important information for transplant centers to utilize when deciding if an organ donor would be appropriate for their recipient. Currently, the United States United Network for Organ Sharing (UNOS) instructs that six antigen-matched organs be given elevated priority. While only a small portion of deceased donor kidneys are well matched, this assay would help provide additional information about the patient typing and potential protein expression that will allow individual centers to risk stratify their candidates for the organ offer with this additional information. The assay demonstrates reliable HLA typing performance even with samples that exhibit a high degree of fragmentation. This has major implications for the transplant laboratory, which often receives patient samples that are not of the highest quality but must be utilized due to time constraints or a lack of better patient samples. Additionally, using this method, it might be possible to provide typing for donors from FFPE slides or tissue samples that display a high degree of fragmentation. Utilizing RNA and the application of UMI counting to quantify the mRNA HLA expression, the data demonstrate the variability of individual HLA allele groups among individuals. The assay results may be essential in both solid organ and hematopoietic stem cell transplants as well other areas of medicine in providing additional information to further evaluate and risk-stratify donor and recipient pairs.

The presently disclosed study is limited in that it used peripheral blood cells for HLA transcript expression. As described earlier herein, this is likely why there is an increased amount of class I mRNA transcripts compared to class II transcripts. There is no way to adequately predict how the cellular components of each patient influenced the mRNA transcript levels calculated, but this likely influenced the transcript expression of each patient. A final limitation was that the presently disclosed study was restricted to only a small cohort of patients.

### REFERENCES

All references cited in the instant disclosure, including but not limited to all patents, patent applications and publications thereof, scientific journal articles, and database entries (including but not limited to GENBANK® biosequence database entries and all annotations available therein) are incorporated herein by reference in their entireties to the extent that they supplement, explain, provide a background for, or teach methodology, techniques, and/or compositions employed herein.
Apps et al. (2013) Influence of HLA-C expression level on HIV control. Science 340 (6128):87-91.
Badders et al. (2015) Variable HLA expression on deceased donor lymphocytes: Not all crossmatches are created equal. Human Immunology 76 (11):795-800.
Barsakis et al. (2019). Complete nucleotide sequence characterization of DRB5 alleles reveals a homogeneous allele group that is distinct from other DRB genes. Human Immunology 80(7):437-448.
Bettens et al. (2014) High-allelic variability in HLA-C mRNA expression: association with HLA-extended haplotypes. Genes & Immunity 15 (3):176-181.
Bettinotti et al. (2003) New HLA-A, -B, and -C locus-specific primers for PCR amplification from cDNA: application in clinical immunology. Journal of Immunological Methods 279 (1-2):143-148.
Chen et al. (2018) UMI-count modeling and differential expression analysis for single-cell RNA sequencing. Genome Biology 19 (1):1-17.
Cornaby & Weimer (2020). HLA Typing by Next-Generation Sequencing. Advances in Molecular Pathology 3:199-205.
Cornaby et al. (2021). Next-generation sequencing and clinical histocompatibility testing. Human Immunology 82(11):829-837.
Darby et al. (2020) scHLAcount: allele-specific HLA expression from single-cell gene expression data. Bioinformatics 36 (12):3905-3906.
Dendrou et al. (2018) HLA variation and disease. Nature Reviews Immunology 18 (5):325-339.
De Santis et al. (2020) Rapid high-resolution HLA genotyping by MinION Oxford nanopore sequencing for deceased donor organ allocation. HLA 96 (2):141-162.
Emery et al. (1993) The different level of expression of HLA-DRB1 and -DRB3 genes is controlled by conserved isotypic differences in promoter sequence. Human Immunology 38 (2):137-147.
Faner et al. (2010) Reassessing the role of HLA-DRB3 T-cell responses: evidence for significant expression and complementary antigen presentation. European Journal of Immunology 40 (1):91-102.
Garcia-Sanchez et al. (2020) The shared epitope phenomenon—A potential impediment to virtual crossmatch accuracy. Clinical Transplantation 34 (8):e13906.
Gutierrez-Arcelus et al. (2020) Allele-specific expression changes dynamically during T cell activation in HLA and other autoimmune loci. Nature Genetics 52 (3):247-253.
Hughes et al. (2020) Quantification of Allele-Specific HLA Expression with Nanopore Long-Read Sequencing. Blood 136 (Supplement 1):42-43.
Islam et al. (2014) Quantitative single-cell RNA-seq with unique molecular identifiers. Nature Methods 11 (2):163-166.
Jain et al. (2018) Nanopore sequencing and assembly of a human genome with ultra-long reads. Nature Biotechnology 36 (4):338-345.
Johansson et al. (2021) HLA RNA Sequencing With Unique Molecular Identifiers Reveals High Allele-Specific Variability in mRNA Expression. Frontiers in Immunology 12:629059.
Kivioja et al. (2011) Counting absolute numbers of molecules using unique molecular identifiers. Nature Methods 9 (1):72-74.
Kou et al. (2016) Benefits and Challenges with Applying Unique Molecular Identifiers in Next Generation Sequencing to Detect Low Frequency Mutations. PLOS ONE 11 (1):e0146638.
Lang et al. (2018) Full-Length HLA Class I Genotyping with the MinION Nanopore Sequencer. Methods Mol Biol 1802:155-162.
Lank et al. (2012) Ultra-high resolution HLA genotyping and allele discovery by highly multiplexed cDNA amplicon pyrosequencing. BMC Genomics 13:378.
le Morvan et al. (2001) HLA-A and HLA-B transcription decrease with ageing in peripheral blood leucocytes. Clinical and Experimental Immunology 125 (2):245-250.
Levin et al. (2020) Optimization for Sequencing and Analysis of Degraded FFPE-RNA Samples. J Vos Exp (160):e61060.
Li (2018). Minimap2: pairwise alignment for nucleotide sequences. Bioinformatics 34(18):3094-3100.
Lind et al. (2010) Next-generation sequencing: the solution for high-resolution, unambiguous human leukocyte antigen typing. Hum Immunol 71 (10):1033-1042.
Liu & Berry (2020) Rapid High-Resolution Typing of Class I HLA Genes by Nanopore Sequencing. Methods in Molecular Biology 2120:93-99.
Liu et al. (2018a) Accurate Typing of Human Leukocyte Antigen Class I Genes by Oxford Nanopore Sequencing. J Mol Diagn 20 (4):428-435.
Liu et al. (2018b) Accurate Typing of Human Leukocyte Antigen Class I Genes by Oxford Nanopore Sequencing. The Journal of Molecular Diagnostics 20 (4):428-435.
Liu et al. (2021) High-resolution HLA typing by long reads from the R10.3 Oxford nanopore flow cells. Human Immunology 82 (4):288-295.
Metzker (2010) Sequencing technologies—the next generation. Nat Rev Genet 11 (1):31-46.
Montgomery et al. (2019) Using Nanopore Whole-Transcriptome Sequencing for Human Leukocyte Antigen Genotyping and Correlating Donor Human Leukocyte Antigen Expression with Flow Cytometric Crossmatch Results. The Journal of Molecular Diagnostics 22(1):101-110.
Mosbruger et al. (2020) Utilizing nanopore sequencing technology for the rapid and comprehensive characterization of eleven HLA loci; addressing the need for deceased donor expedited HLA typing. Human Immunology 81 (8):413-422.
Petersdorf et al. (2015) High HLA-DP Expression and Graft-versus-Host Disease. N Engl J Med 373 (7):599-609.
Resource Centre (2017) WWW Document. Oxford Nanopore Technologies. URL http://londoncallingconf.co.uk/resource-centre (accessed 4/22/2022).
Rognes et al. (2016) VSEARCH: A versatile open source tool for metagenomics. PeerJ 2016 (10):e2584.
Shi et al. (2021) Bias in RNA-seq Library Preparation: Current Challenges and Solutions. BioMed Research International 2021:6647597.
Voorter et al. (1997). The absence of DR51 in a DRB5-positive individual DR2ES is caused by a null allele (DRB5*0108N). Tissue Antigens 50(4):326-333.
Weimer et al. (2016) Performance Characteristics and Validation of Next-Generation Sequencing for Human Leucocyte Antigen Typing. Journal of Molecular Diagnostics 18 (5):668-675.
Weimer & Newhall (2019) Development of data-driven models for the flow cytometric crossmatch. Human Immunology 80 (12):983-989.
Wick et al. (2018) Deepbinner: Demultiplexing barcoded Oxford Nanopore reads with deep convolutional neural networks. PLOS Computational Biology 14 (11):e1006583.
Xu et al. (2020) NanoSPC: a scalable, portable, cloud compatible viral nanopore metagenomic data processing pipeline. Nucleic Acids Research, 48 (W1):W366-W371.

TABLE 1
Cohort Sociodemographic Characteristics

| Characteristic | Values (N = 18) | |
|---|---|---|
| Age, Median (Range) | 53 | (25-67) |

Gender, Count (%)

| | | |
|---|---|---|
| Female | 11 | (61.1) |
| Male | 7 | (39.9) |

Race, Count (%)

| | | |
|---|---|---|
| African American | 11 | (61.1) |
| European American | 6 | (33.3) |
| Unknown | 1 | (5.6) |

TABLE 2
Basic Primer Sequences for GSP and UVP Primers

| | Length | |
|---|---|---|
| Primer Name | (bp) | Primer Sequence* |
| GSP UMI Forward | 71-76 | gtatcgtgtagagactgcgtagg |
| | | TTTVVVVTTVVVVTTVVVVTTVV |
| | | VVTTTnnnnnnnnnnnnnnnnnn |
| | | nn** |
| GSP UMI Reverse | 70-75 | AGTGATCGAGTCAGTGCGAGTGT |
| | | TTVVVVTTVVVVTTVVVVTTVVV |
| | | VTTTnnnnnnnnnnnnnnnnnnn |
| | | n** |
| UVP Forward | 60 | GGTGCTGAAGAAAGTTGTCGGTG |
| | | TCTTTGTGTTAACCgtatcgtgt |
| | | agagactgcgtagg*** |
| UVP Reverse | 59 | GGTGCTGAAGAAAGTTGTCGGTG |
| | | TCTTTGTGTTAACCAGTGATCGA |
| | | GTCAGTGCGAGTG**** |

*V = A, C, or G (i.e., not T)
**the nnnnnnnnnnnnnnnnnnnn sequence in the forward and reverse GSP UMI primers corresponds to pairs of forward and reverse primer sequences listed in Table below.
**the sequence in italics corresponds to an additional primer binding site that permits amplification along with the sequence below in bold following attachment of the HLA-specific UMI primers.
****the sequence in bold corresponds to an additional primer binding site that permits amplification along with the sequence above in italics following attachment of the HLA- specific UMI primers.

TABLE 3
Primer Sequences for HLA-specific PCR Amplifications

| HLA | Listed 5′-3′ | | |
|---|---|---|---|
| TARGET | FORWARD (5′-3′)* | REVERSE (5′-3′)** | PMID |
| A | CCAGACGCCGAGGATGGCC | ACACAAGGCAGCTGTCTCACA | 12969555 |
| | (SEQ ID NO: 1) | (SEQ ID NO: 2) | |
| B | AATCTCCTCAGACGCCGA | CAAGCTGTGAGAGACACATCAG | |
| | (SEQ ID NO: 3) | (SEQ ID NO: 4) | |
| C | GCCGAGATGCGGGTCATGG | ACACAGGCAGCTGTCTCAGG | |
| | (SEQ ID NO: 5) | (SEQ ID NO: 6) | |
| DRB1/ | CCCCTGGTCCTGTCCTG | CAAGGAAGAGCAGGCCCAGCA | 17174751/ |
| 3/4/5 | (SEQ ID NO: 7) | (SEQ ID NO: 8) | 22866951 |
| DPA1 | ATGCGCCCRGAAGACAGAATGT | CCCCTGGGCCCGGGG | 22866951 |
| | (SEQ ID NO: 9) | (SEQ ID NO: 10) | |
| DPB1 | CCTTCTTTTCCTGACTGCAG | TGYTAACRAAACACAGCAAATGC | 22224408 |
| | (SEQ ID NO: 11) | (SEQ ID NO: 12) | |
| DQA1 | ATGATCCTAAACAAAGCTCTG | GGTGTCTGGAAGCACCAACTG | 22866951 |
| | (SEQ ID NO: 13) | (SEQ ID NO: 14) | |
| DQB1 | GAARAAGKCTTTGCGGATCCC | AGTCTCAGGAGTCAGTGCAG | 22866951 |
| | (SEQ ID NO: 15) | (SEQ ID NO: 16) | |

*the sequences listed for the forward primers correspond to the nnnnnnnnnnnnnnnnnnnn sequences in the GSP UMI Forward primers listed in Table 2.
**the sequences listed for the reverse primers correspond to the nnnnnnnnnnnnnnnnnnnn sequences in the GSP UMI Reverse primers listed in Table 2.

It will be understood that various details of the presently disclosed subject matter can be changed without departing from the scope of the presently disclosed subject matter. Furthermore, the foregoing description is for the purpose of illustration only, and not for the purpose of limitation.

TABLE 4
RNA-Seq HLA Typing Results Using NGSEngine

| | HLA-A | | HLA-B | | HLA-C | | HLA-DPA1 | | HLA-DPB1 | |
|---|---|---|---|---|---|---|---|---|---|---|
| | Refer- | NGSEn- | Refer- | NGSEn- | Refer- | NGSEn- | Refer- | NGSEn- | Refer- | NGSEn- |
| Sample ID | ence | gine | ence | gine | ence | gine | ence | gine | ence | gine |
| cDNA001 Mar. 25, 2021 | 02:01:01 | 02:01:01 | 35:01:01 | 35:01:01 | 03:04:01 | 03:04:01 | 01:03:01 | 01:03:01 | 02:01:02 | 02:01:02 |
| | 11:01:01 | 11:01:01 | 40:01:02 | 40:01:02 | 04:01:01 | 04:01:01 | — | — | 03:01:01 | 03:01:01 |
| cDNA057 Mar. 25, 2021 | 02:01:01 | 02:01:01 | 07:06:01 | 07:06:01 | 15:05:02 | 15:05:02 | 01:03:01 | 01:03:01 | 04:01:01 | 04:01:01 |
| | 03:01:01 | 03:01:01 | 44:03:01 | 44:03:01 | 16:01:01 | 16:01:01 | — | — | — | — |
| cDNA058 Mar. 25, 2021 | 03:01:01 | 03:01:01 | 53:01:01 | 53:01:01 | 04:01:01 | 04:01:01 | 01:03:01 | 01:03:01 | 04:01:01 | 04:01:01 |
| | — | — | 57:02:01 | 57:02:01 | 18:02:01 | 18:02:01 | 03:01:01 | 03:01:01 | 105:01:0 | 105:01:01 |
| cDNA061 Aug. 9, 2021 | 11:01:01 | 11:01:01 | 40:01:02 | 40:01:02 | 03:03:01 | 03:03:01 | 01:03:01 | 01:03:01 | 04:01P | 04:01:01 |
| | 24:02:01 | 24:02:01 | 55:01:01 | 55:01:01 | 03:04:01 | 03:04:01 | 02:01:01 | 02:01:01 | 10:01P | 10:01:01 |
| cDNA062 Mar. 25, 2021 | 30:01:01 | 30:01:01 | 35:01:01 | 35:01:01 | 04:01:01 | 04:01:01 | 01:03:01 | 01:03:01 | 04:01P | 04:01:01 |
| | 30:02:01 | 30:02:01 | 42:01:01 | 42:01:01 | 17:01:01 | 17:01:01 | — | — | 03:01P | 104:01:01 |
| cDNA068 May 6, 2021 | 02:01:01 | 02:01:01 | 07:02:01 | 07:02:01 | 07:02:01 | 07:02:01 | 01:03:01 | 01:03:01 | 02:01:02 | 02:01:02 |
| | 03:01:01 | 03:01:01 | 44:03:01 | 44:03:01 | 16:01:01 | 16:01:01 | 02:01:01 | 02:01:01 | 11:01:01 | 11:01:01 |
| cDNA069 May 6, 2021 | 30:01:01 | 30:01:01 | 42:01:01 | 42:01:01 | 06:02:01 | 06:02:01 | 02:01:01 | 02:01:01 | 01:01P | 01:01:01 |
| | 68:01:01 | 68:01:01 | 58:02:01 | 58:02:01 | 17:01:01 | 17:01:01 | 02:02:02 | 02:02:02 | 11:01P | 11:01:01 |
| cDNA070 Jul. 8, 2021 | 23:01:01 | 23:01:01 | 44:03:01 | 44:03:01 | 04:01:01 | 04:01:01 | 01:03:01 | 01:03:01 | 17:01:01 | 17:01:01 |
| | 26:01:01 | 26:01:01 | — | — | — | — | 02:01:01 | 02:01:01 | 18:01:01 | 18:01:01 |
| cDNA071 May 6, 2021 | 01:01:01 | 01:01:01 | 08:01:01 | 08:01:01 | 07:01:01 | 07:01:01 | 01:03:01 | 01:03:01 | 01:01:01 | 01:01:01 |
| | 30:02:01 | 30:02:01 | 47:03 | 47:03 | 07:18:01 | 07:18:01 | 02:02:02 | 02:02:02 | 06:01:01 | 06:01:01 |
| cDNA072 May 6, 2021 | 03:01:01 | 03:01:01 | 07:02:01 | 07:02:01 | 04:01:01 | 04:01:01 | 02:02:02 | 02:02:02 | 01:01:01 | 01:01:01 |
| | 23:01:01 | 23:01:01 | 44:03:01 | 44:03:01 | 07:02:01 | 07:02:01 | 03:01:01 | 03:01:01 | 105:01:01 | 105:01:01 |
| cDNA073 Jul. 8, 2021 | 03:01:01 | 03:01:01 | 35:01:01 | 35:01:01 | 04:01:01 | 04:01:01 | 01:03:01 | 01:03:01 | 17:01:01 | 17:01:01 |
| | 30:01:01 | 30:01:01 | 44:03:01 | 44:03:01 | — | — | 02:01:01 | 02:01:01 | 18:01:01 | 18:01:01 |
| cDNA074 Jul. 8, 2021 | 23:01:01 | 23:01:01 | 44:03:01 | 44:03:01 | 04:01:01 | 04:01:01 | 01:03:01 | 01:03:01 | 02:01:02 | 02:01:02 |
| | 36:01 | 36:01 | 53:01:01 | 53:01:01 | — | — | — | — | — | — |
| cDNA075 Jul. 8, 2021 | 23:01:01 | 23:01:01 | 45:01:01 | 45:01:01 | 06:02:01 | 06:02:01 | 01:03:01 | 01:03:01 | 01:01:01 | 01:01:01 |
| | 23:17 | 23:17 | — | — | 16:01:01 | 16:01:01 | 02:01:08 | 02:01:08 | 02:01:19 | 02:01:19 |
| cDNA076 Jul. 8, 2021 | 68:02:01 | 68:02:01 | 15:03:01 | 15:03:01 | 02:10:01 | 02:10:01 | 02:01:01 | 02:01:01 | 05:01:01 | 05:01:01 |
| | 74:01:01 | 74:01:01 | 15:10:01 | 15:10:01 | 03:04:02 | 03:04:02 | 02:02:02 | 02:02:02 | 11:01:01 | 11:01:01 |
| cDNA077 Aug. 9, 2021 | 02:01:01 | 02:01:01 | 49:01:01 | 49:01:01 | 07:01:02 | 07:01:02 | 01:03:01 | 01:03:01 | 04:02P | 105:01:01 |
| | 03:01:01 | 03:01:01 | 51:01:01 | 51:01:01 | 14:02:01 | 14:02:01 | 03:01:01 | 03:01:01 | — | — |
| cDNA078 Aug. 5, 2021 | 02:01:01 | 02:01:01 | 42:01:01 | 42:01:01 | 05:01:01 | 05:01:01 | 01:03:01 | 01:03:01 | 04:02:01 | 04:02:01 |
| | 30:01:01 | 30:01:01 | 44:02:01 | 44:02:01 | 17:01:01 | 17:01:01 | — | — | 18:01:01 | 18:01:01 |
| cDNA079 Aug. 5, 2021 | 01:01:01 | 01:01:01 | 37:01:01 | 37:01:01 | 03:04:01 | 03:04:01 | 01:03:01 | 01:03:01 | 01:01:01 | 01:01:01 |
| | 02:01:01 | 02:01:01 | 40:01:02 | 40:01:02 | 06:02:01 | 06:02:01 | 02:01:02 | 02:01:02 | 04:01:01 | 04:01:01 |
| cDNA082 Aug. 11, 2021 | 11:01:01 | 11:01:01 | 15:10:01 | 15:10:01 | 01:02:01 | 01:02:01 | 01:03:01 | 01:03:01 | 04:01:01 | 04:01:01 |
| | 33:03:01 | 33:03:01 | 56:01:01 | 56:01:01 | 03:02:02 | 03:02:02 | — | — | 18:01:01 | 18:01:01 |

| | HLA-DQA | | HLA-DQB1 | | HLA-DRB1 | | HLA-DRB3/4/5 | |
|---|---|---|---|---|---|---|---|---|
| | Refer- | NGSEn- | Refer- | NGSEn- | Refer- | NGSEn- | Refer- | NGSEn- |
| Sample ID | ence | gine | ence | gine | ence | gine | ence | gine |
| cDNA001 Mar. 25, 2021 | 01:01:01 | 01:01:01 | 05:01:01 | 05:01:01 | 01:01:01 | 01:01:01 | | |
| | 01:04:01 | 01:04:01 | 05:03:01 | 05:03:01 | 14:54:01 | 14:54:01 | 3*02:02:01 | 3*02:02:01 |
| cDNA057 Mar. 25, 2021 | 02:01:01 | 02:01:01 | 02:02:01 | 02:02:01 | 11:02:01 | 11:02:01 | 3*02:02:01 | 3*02:02:01 |
| | 05:05:01 | 01:01:01 | 03:19:01 | 03:19:01 | 07:01:01 | 07:01:01 | 4*01:01:01 | 4*01:01:01 |
| cDNA058 Mar. 25, 2021 | 03:03:01 | 03:03:01 | 02:02:01 | 02:02:01 | 11:01:02 | 11:01:02 | 3*02:02:01 | 3*02:02:01 |
| | 05:05:01 | 05:05:01 | 03:19:01 | 03:19:01 | — | — | | |
| cDNA061 Aug. 9, 2021 | 03:01:01 | 03:01:01 | 03:01:01 | 03:01:01 | 11:01:01 | 11:01:01 | 3*02:02:01 | 3*02:02:01 |
| | 05:05:01 | 05:05:01 | 03:02:01 | 03:02:01 | 04:04:01 | 04:04:01 | 4*01:03:01 | 4*01:03:01 |
| cDNA062 Mar. 25, 2021 | 01:02:01 | 01:02:01 | 03:19:01 | 03:19:01 | 11:02:01 | 11:02:01 | 3*02:02:01 | 3*02:02:01 |
| | 05:05:01 | 05:05:01 | 06:09:01 | 06:09:01 | 13:02:01 | 13:02:01 | 3*03:01:01 | 3*03:01:01 |
| cDNA068 May 6, 2021 | 02:01:01 | 02:01:01 | 02:02:01 | 02:02:01 | 04:01:01 | 04:01:01 | 4*01:01:01 | 4*01:01:01 |
| | 03:01:01 | 03:01:01 | 03:02:01 | 03:02:01 | 07:01:01 | 07:01:01 | 4*01:03:01 | 4*01:03:01 |
| cDNA069 May 6, 2021 | 01:05:01 | 01:05:01 | 04:02:01 | 04:02:01 | 03:02:01 | 03:02:01 | 3*01:62:01 | 3*01:62:01 |
| | 04:01:01 | 04:01:01 | 05:01:01 | 05:01:01 | 12:01:01 | 12:01:01 | 3*02:02:01 | 3*02:02:01 |
| cDNA070 Jul. 8, 2021 | 01:02:01 | 01:02:01 | 05:02:01 | 05:02:01 | 13:02:01 | 13:02:01 | 3*03:01:01 | 3*03:01:01 |
| | — | — | 06:09:01 | 06:09:01 | 13:31 | 13:31 | | |
| cDNA071 May 6, 2021 | 05:01:01 | 05:01:01 | 02:01:01 | 02:01:01 | 03:01:01 | 03:01:01 | 3*01:01:02 | 3*01:01:02 |
| | — | — | — | — | — | — | 3*02:02:01 | 3*02:02:01 |
| cDNA072 May 6, 2021 | 01:02:01 | 01:02:01 | 03:19:01 | 03:19:01 | 15:03:01 | 15:03:01 | 5*01:01:01 | 5*01:01:01 |
| | 05:05:01 | 05:05:01 | 06:02:01 | 06:02:01 | 11:02:01 | 11:02:01 | 3*02:02:01 | 3*02:02:01 |
| cDNA073 Jul. 8, 2021 | 01:02:01 | 01:02:01 | 02:02:01 | 02:02:01 | 07:01:01 | 07:01:01 | 4*01:03:01 | 4*01:03:01 |
| | 02:01:01 | 02:01:01 | 06:02:01 | 06:02:01 | 15:03:01 | 15:03:01 | 5*01:01:01 | 5*01:01:01 |
| cDNA074 Jul. 8, 2021 | 01:02:01 | 01:02:01 | 02:02:01 | 02:02:01 | 11:01:02 | 11:01:02 | 3*02:02:01 | 3*02:02:01 |
| | 02:01:01 | 02:01:01 | 06:02:01 | 06:02:01 | 07:01:01 | 07:01:01 | 4*01:01:01 | 4*01:01:01 |
| cDNA075 Jul. 8, 2021 | 01:02:01 | 01:02:01 | 02:01:01 | 02:01:01 | 03:01 | 03:01:01 | 3*02:02:01 | 3*02:02:01 |
| | 05:01:01 | 05:01:01 | 05:02:01 | 05:02:01 | 11:01 | 11:01:02 | 3*03:01:01 | 3*03:01:01 |
| cDNA076 Jul. 8, 2021 | 01:01:02 | 01:01:02 | 02:02:01 | 02:02:01 | 07:01:01 | 07:01:01 | 4*01:01:01 | 4*01:01:01 |
| | 03:03:01 | 03:03:01 | 05:01:01 | 05:01:01 | 01:02:01 | 01:02:01 | | |
| cDNA077 Aug. 9, 2021 | 01:01:01 | 01:01:01 | 03:19:01 | 03:19:01 | 15:02P | 15:02:01 | 5*01:08:01N | |
| | 04:01:02 | 04:01:02 | 05:01:24 | 05:01:24 | 08:04:01 | 08:04:01 | | |
| cDNA078 Aug. 5, 2021 | 01:02:01 | 01:02:01 | 03:01:01 | 03:01:01 | 04:01:01 | 04:01:01 | 4*01:03:01 | 4*01:03:01 |
| | 03:03:01 | 03:03:01 | 06:02:01 | 06:02:01 | 15:03:01 | 15:03:01 | | |
| cDNA079 Aug. 5, 2021 | 01:02:01 | 01:02:01 | 05:01:01 | 05:01:01 | 10:01:01 | 10:01:01 | | |
| | 01:05:01 | 01:05:01 | 06:02:01 | 06:02:01 | 15:01:01 | 15:01:01 | 5*01:01:01 | 5*01:01:01 |
| cDNA082 Aug. 11, 2021 | 01:01:01 | 01:01:01 | 05:01:01 | 05:01:01 | 01:01:01 | 01:01:01 | | |
| | 01:02:01 | 01:02:01 | 06:02:01 | 06:02:01 | 15:03:01 | 15:03:01 | 5*01:01:01 | 5*01:01:01 |

1. A method for rapid, multiplex determination of HLA status of a cell, tissue, organ, or subject, the method comprising:
(a) providing a sample from the cell, tissue, or organ that comprises mRNA derived from an HLA gene product;
(b) reverse transcribing the mRNA to produce a pool of cDNAs, wherein the pool of cDNAs comprises a plurality of HLA-specific cDNAs;
(c) amplifying the cDNAs that result from reverse transcription of HLA-specific mRNAs to produce a pool of HLA-specific cDNAs, wherein:
(i) the HLA-specific mRNAs are transcription products of one or more of HLA-A, HLA-B, HLA-C, HLA-E, HLA-F, HLA-G, HLA-H, HLA-DRB1, HLA-DRB3, HLA-DRB4, HLA-DRB5, HLA-DQA1, HLA-DQB1, HLA-DPA1, HLA-DPB1, MICA, and MICB loci, optionally for each of at least HLA-A, HLA-B, HLA-C, HLA-DRB1/3/4/5, HLA-DQA1, and HLA-DQB1;
(ii) the amplifying comprises an enrichment step and an amplification step, wherein the amplification step is performed subsequent to the enrichment step; and
(iii) the amplification employs HLA-specific primers that comprise unique molecular identifiers (UMIs) such that the HLA-specific cDNAs are each uniquely coded; and
(d) determining the sequence of each HLA-specific cDNAs,
whereby the HLA status of the cell, tissue, organ, or subject is determined.

2. The method of claim 1, wherein the sample is a blood sample.

3. The method of claim 1, wherein the sample is isolated from a deceased donor.

4. The method of claim 1, wherein the amplifying identifies each of HLA-A, -B, -C, -DRB1/3/4/5, -DQA1, and -DQB1 if present in the sample.

5. The method of claim 1, wherein the HLA-specific primers comprise HLA-specific sequences, and further wherein the HLA-specific sequences comprise sequences selected from the group consisting of SEQ ID NO: 1 and SEQ ID NO: 2 for HLA-A, SEQ ID NO: 3 and SEQ ID NO: 4 for HLA-B, SEQ ID NO: 5 and SEQ ID NO: 6 for HLA-C, SEQ ID NO: 7 and SEQ ID NO: 8 for HLA-DRB1/3/4/5, SEQ ID NO: 9 SEQ ID NO: 10 for HLA-DPA1, SEQ ID NO: 11 SEQ ID NO: 12 for HLA-DPB1, SEQ ID NO: 13 and SEQ ID NO: 14 for HLA-DQA1, and SEQ ID NO: 15 and SEQ ID NO: 16 for HLA-DQB1.

6. The method of claim 1, wherein the determining the sequence of each HLA-specific cDNAs step comprises use of an Oxford Nanopore Technologies flow cell, optionally selected from the group consisting of an R10.3 flow cell, an R10.4 flow cell, an R9.4 flow cell, or any updated version thereof, and a Flongle flow cell, and an Oxford Nanopore Technologies MinION R10.3 Flow Cell sequencer, optionally selected from the group consisting of a MinION sequencer, a MinION Mk1C sequencer, a Promethion sequencer, a Gridion sequencer, a MinION Mk1D sequencer, a SmidgION sequencer, and a Plongle sequencer.

7. The method of claim 1, wherein the cell, tissue, or organ is a donor cell, tissue, or organ is meant for transfer to a recipient and/or is derived from a donor cell, tissue, or organ meant for transfer to a recipient.

8. The method of claim 1, wherein the determination of HLA status of the cell, tissue, organ, or subject is intended for use in target point of care viral load testing to provide absolute viral counts, optionally wherein the virus is an HIV or a CMV, or is intended for use in diagnosis of an immune-associated disease, disorder, or condition, optionally wherein the immune-associated disease, disorder, or condition is Bechet Disease, Celiac Disease, or ankylosing spondylitis.

9. A method for genotyping a donor cell, tissue, and/or organ meant for transplantation into a recipient and/or for genotyping the recipient thereof, the method comprising:
(a) providing a donor cell, tissue, and/or organ meant for transplantation into a recipient and/or a cell, tissue, and/or organ from the recipient thereof that comprises mRNA derived from an HLA gene product;
(b) reverse transcribing the mRNA to produce a pool of cDNAs, wherein the pool of cDNAs comprises a plurality of HLA-specific cDNAs;
(c) amplifying the cDNAs that result from reverse transcription of HLA-specific mRNAs to produce a pool of HLA-specific cDNAs, wherein:
(i) the HLA-specific mRNAs are transcription products of one or more of HLA-A, HLA-B, HLA-C, HLA-E, HLA-F, HLA-G, HLA-H, HLA-DRB1, HLA-DRB3, HLA-DRB4, HLA-DRB5, HLA-DQA1, HLA-DQB1, HLA-DPA1, HLA-DPB1, MICA, and MICB loci, optionally for each of at least HLA-A, HLA-B, HLA-C, HLA-DRB1/3/4/5, HLA-DQA1, and HLA-DQB1;
(ii) the amplifying comprises an enrichment step and an amplification step, wherein the amplification step is performed subsequent to the enrichment step; and
(iii) the amplification employs HLA-specific primers that comprise unique molecular identifiers (UMIs) such that the HLA-specific cDNAs are each uniquely coded; and
(d) determining the sequence of each HLA-specific cDNAs,
whereby the donor cell, tissue, and/or organ meant for transplantation into a recipient and/or the recipient thereof is genotyped.

10. The method of claim 9, wherein the donor cell, tissue, and/or organ is a cadaveric cell, tissue, and/or organ.

11. The method of claim 9, wherein the cell, tissue, and/or organ from the recipient is any isolated cell, tissue, and/or organ that comprises mRNA derived from an HLA gene product from the recipient.

11. The method of claim 9, wherein the amplifying identifies each of HLA-A, -B, -C, -DRB1/3/4/5, -DQA1, and -DQB1 if present in the sample.

12. The method of claim 9, wherein the HLA-specific primers comprise HLA-specific sequences, and further wherein the HLA-specific sequences comprise sequences selected from the group consisting of SEQ ID NO: 1 and SEQ ID NO: 2 for HLA-A, SEQ ID NO: 3 and SEQ ID NO: 4 for HLA-B, SEQ ID NO: 5 and SEQ ID NO: 6 for HLA-C, SEQ ID NO: 7 and SEQ ID NO: 8 for HLA-DRB1/3/4/5, SEQ ID NO: 9 SEQ ID NO: 10 for HLA-DPA1, SEQ ID NO: 11 SEQ ID NO: 12 for HLA-DPB1, SEQ ID NO: 13 and SEQ ID NO: 14 for HLA-DQA1, and SEQ ID NO: 15 and SEQ ID NO: 16 for HLA-DQB1.

13. The method of claim 9, wherein the determining the sequence of each HLA-specific cDNAs step comprises use of an Oxford Nanopore Technologies flow cell, optionally selected from the group consisting of an R10.3 flow cell, an R10.4 flow cell, an R9.4 flow cell, or any updated version thereof, and a Flongle flow cell, and an Oxford Nanopore Technologies MinION R10.3 Flow Cell sequencer, optionally selected from the group consisting of a MinION sequencer, a MinION Mk1C sequencer, a Promethion sequencer, a Gridion sequencer, a MinION Mk1D sequencer, a SmidgION sequencer, and a Plongle sequencer.


SEQUENCE LISTING (36 sequences)

SEQ ID NO: 1 - DNA, 19 bp, Homo sapiens
  source 1..19: mol_type = other DNA
ccagacgccgaggatggcc

SEQ ID NO: 2 - DNA, 21 bp, Homo sapiens
  source 1..21: mol_type = other DNA
acacaaggcagctgtctcaca

SEQ ID NO: 3 - DNA, 18 bp, Homo sapiens
  source 1..18: mol_type = other DNA
aatctcctcagacgccga

SEQ ID NO: 4 - DNA, 22 bp, Homo sapiens
  source 1..22: mol_type = other DNA
caagctgtgagagacacatcag

SEQ ID NO: 5 - DNA, 19 bp, Homo sapiens
  source 1..19: mol_type = other DNA
gccgagatgcgggtcatgg

SEQ ID NO: 6 - DNA, 20 bp, Homo sapiens
  source 1..20: mol_type = other DNA
acacaggcagctgtctcagg

SEQ ID NO: 7 - DNA, 17 bp, Homo sapiens
  source 1..17: mol_type = other DNA
cccctggtcctgtcctg

SEQ ID NO: 8 - DNA, 21 bp, Homo sapiens
  source 1..21: mol_type = other DNA
caaggaagagcaggcccagca

SEQ ID NO: 9 - DNA, 22 bp, Homo sapiens
  source 1..22: mol_type = other DNA
  variation 9: note = r = a or g
atgcgcccrgaagacagaatgt

SEQ ID NO: 10 - DNA, 15 bp, Homo sapiens
  source 1..15: mol_type = other DNA
cccctgggcccgggg

SEQ ID NO: 11 - DNA, 20 bp, Homo sapiens
  source 1..20: mol_type = other DNA
ccttcttttcctgactgcag

SEQ ID NO: 12 - DNA, 23 bp, Homo sapiens
  source 1..23: mol_type = other DNA
  variation 3: note = y = c or t
  variation 8: note = r = a or g
tgytaacraaacacagcaaatgc

SEQ ID NO: 13 - DNA, 21 bp, Homo sapiens
  source 1..21: mol_type = other DNA
atgatcctaaacaaagctctg

SEQ ID NO: 14 - DNA, 21 bp, Homo sapiens
  source 1..21: mol_type = other DNA
ggtgtctggaagcaccaactg

SEQ ID NO: 15 - DNA, 21 bp, Homo sapiens
  source 1..21: mol_type = other DNA
  variation 4: note = r is a or g
  variation 8: note = k is g or t
gaaraagkctttgcggatccc

SEQ ID NO: 16 - DNA, 20 bp, Homo sapiens
  source 1..20: mol_type = other DNA
agtctcaggagtcagtgcag

SEQ ID NO: 17 - DNA, 71 bp, synthetic construct
  source 1..71: mol_type = other DNA
  variation 51..71: note = nnnnnnnnnnnnnnnnnnnn is any of SEQ ID NOs: 1, 3, 5, 7, 9, 11, 13, and 15
  variation 27..48: note = v= a, c, or g
gtatcgtgtagagactgcgtaggtttvvvvttvvvvttvvvvttvvvvtttnnnnnnnnnnnnnnnnnnnn

SEQ ID NO: 18 - DNA, 70 bp, synthetic construct
  source 1..70: mol_type = other DNA
  variation 27..48: note = v = a, c, or g
gtatcgtgtagagactgcgtaggtttvvvvttvvvvttvvvvttvvvvtttccagacgccgaggatggcc

SEQ ID NO: 19 - DNA, 69 bp, synthetic construct
  source 1..69: mol_type = other DNA
  variation 27..48: note = v = a, c, or g
gtatcgtgtagagactgcgtaggtttvvvvttvvvvttvvvvttvvvvtttaatctcctcagacgccga

SEQ ID NO: 20 - DNA, 70 bp, synthetic construct
  source 1..70: mol_type = other DNA
  variation 27..48: note = v = a, c, or g
gtatcgtgtagagactgcgtaggtttvvvvttvvvvttvvvvttvvvvtttgccgagatgcgggtcatgg

SEQ ID NO: 21 - DNA, 68 bp, synthetic construct
  source 1..68: mol_type = other DNA
  variation 27..48: note = v = a, c, or g
gtatcgtgtagagactgcgtaggtttvvvvttvvvvttvvvvttvvvvtttcccctggtcctgtcctg

SEQ ID NO: 22 - DNA, 73 bp, synthetic construct
  source 1..73: mol_type = other DNA
  variation 27..48: note = v = a, c, or g
gtatcgtgtagagactgcgtaggtttvvvvttvvvvttvvvvttvvvvtttatgcgcccrgaagacagaatgt

SEQ ID NO: 23 - DNA, 71 bp, synthetic construct
  source 1..71: mol_type = other DNA
  variation 27..48: note = v = a, c, or g
gtatcgtgtagagactgcgtaggtttvvvvttvvvvttvvvvttvvvvtttccttcttttcctgactgcag

SEQ ID NO: 24 - DNA, 72 bp, synthetic construct
  source 1..72: mol_type = other DNA
  variation 27..48: note = v = a, c, or g
gtatcgtgtagagactgcgtaggtttvvvvttvvvvttvvvvttvvvvtttatgatcctaaacaaagctctg

SEQ ID NO: 25 - DNA, 72 bp, synthetic construct
  source 1..72: mol_type = other DNA
  variation 27..48: note = v = a, c, or g
gtatcgtgtagagactgcgtaggtttvvvvttvvvvttvvvvttvvvvtttgaaraagkctttgcggatccc

SEQ ID NO: 26 - DNA, 70 bp, synthetic construct
  source 1..70: mol_type = other DNA
  variation 51..70: note = nnnnnnnnnnnnnnnnnnnn is any of SEQ ID NOs: 2, 4, 6, 8, 10, 12, 14, and 16
  variation 26..47: note = v = a, c, or g
agtgatcgagtcagtgcgagtgtttvvvvttvvvvttvvvvttvvvvtttnnnnnnnnnnnnnnnnnnnn

SEQ ID NO: 27 - DNA, 71 bp, synthetic construct
  source 1..71: mol_type = other DNA
  variation 26..47: note = v = a, c, or g
agtgatcgagtcagtgcgagtgtttvvvvttvvvvttvvvvttvvvvtttacacaaggcagctgtctcaca

SEQ ID NO: 28 - DNA, 72 bp, synthetic construct
  source 1..72: mol_type = other DNA
  variation 26..47: note = v = a, c, or g
agtgatcgagtcagtgcgagtgtttvvvvttvvvvttvvvvttvvvvtttcaagctgtgagagacacatcag

SEQ ID NO: 29 - DNA, 70 bp, synthetic construct
  source 1..70: mol_type = other DNA
  variation 26..47: note = v = a, c, or g
agtgatcgagtcagtgcgagtgtttvvvvttvvvvttvvvvttvvvvtttacacaggcagctgtctcagg

SEQ ID NO: 30 - DNA, 71 bp, synthetic construct
  source 1..71: mol_type = other DNA
  variation 26..47: note = v = a, c, or g
agtgatcgagtcagtgcgagtgtttvvvvttvvvvttvvvvttvvvvtttcaaggaagagcaggcccagca

SEQ ID NO: 31 - DNA, 65 bp, synthetic construct
  source 1..65: mol_type = other DNA
  variation 26..47: note = v = a, c, or g
agtgatcgagtcagtgcgagtgtttvvvvttvvvvttvvvvttvvvvtttcccctgggcccgggg

SEQ ID NO: 32 - DNA, 73 bp, synthetic construct
  source 1..73: mol_type = other DNA
  variation 26..47: note = v = a, c, or g
agtgatcgagtcagtgcgagtgtttvvvvttvvvvttvvvvttvvvvttttgytaacraaacacagcaaatgc

SEQ ID NO: 33 - DNA, 71 bp, synthetic construct
  source 1..71: mol_type = other DNA
  variation 26..47: note = v = a, c, or g
agtgatcgagtcagtgcgagtgtttvvvvttvvvvttvvvvttvvvvtttggtgtctggaagcaccaactg

SEQ ID NO: 34 - DNA, 70 bp, synthetic construct
  source 1..70: mol_type = other DNA
  variation 26..47: note = v = a, c, or g
agtgatcgagtcagtgcgagtgtttvvvvttvvvvttvvvvttvvvvtttagtctcaggagtcagtgcag

SEQ ID NO: 35 - DNA, 60 bp, synthetic construct
  source 1..60: mol_type = other DNA
ggtgctgaagaaagttgtcggtgtctttgtgttaaccgtatcgtgtagagactgcgtagg

SEQ ID NO: 36 - DNA, 59 bp, synthetic construct
  source 1..59: mol_type = other DNA
ggtgctgaagaaagttgtcggtgtctttgtgttaaccagtgatcgagtcagtgcgagtg